(19) United States
(12) Patent Application Publication
(10) Pub. No.: US 2026/0034350 A1
(43) Pub. Date: Feb. 5, 2026

(54) VOLTAMMETRY TECHNOLOGIES

(71) Applicant: COCHLEAR LIMITED, Macquarie University, NSW (AU)

(72) Inventors: Oliver John RIDLER, Macquarie University (AU); Paul Michael CARTER, Macquarie University (AU)

(21) Appl. No.: 19/099,086

(22) PCT Filed: Jul. 28, 2023
(86) PCT No.: PCT/IB2023/057719

§ 371 Date: Jan. 28, 2025

Related U.S. Application Data
(60) Provisional application No. 63/393,052, filed on Jul. 28, 2022.

(51) Int. Cl. A61N 1/05; A61N 1/36

(52) U.S. Cl. CPC A61N 1/0541; A61N 1/36038; A61N 1/3605

(57) ABSTRACT

A device, such as a medical device, including a portion, such as an implantable portion of the medical device, which portion includes at least one electrode, wherein the portion, which can be an implantable portion, is configured to, while located in a human, obtain data indicative of a state of a metal-electrolyte interface of the at least one electrode.

### CROSS-REFERENCE TO RELATED APPLICATIONS

This application claims priority to U.S. Provisional Application No. 63/393,052, entitled VOLTAMMETRY TECHNOLOGIES, filed on Jul. 28, 2022, naming Oliver John RIDLER as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an embodiment, there is a medical device, comprising an implantable portion of the medical device, the implantable portion including at least one electrode, wherein the implantable portion is configured to, while implanted in a human, obtain data indicative of a state of a metal-electrolyte interface of the at least one electrode.

In an embodiment, there is a medical device, comprising at least one electrode and electronics configured to controllably apply an electrical signal to the electrode, wherein the medical device is configured to enable in vivo obtention of data indicative of a chemical in an environment of the at least one electrode via voltammetry.

In an embodiment, there is a method, comprising obtaining data relating to a phenomenon internal to a human having an electrode implanted in the human and analyzing the obtained data to determine data indicative of a real surface area of the electrode, wherein the action of obtaining data is executed, at the time of obtaining data, non-invasively.

In an embodiment, there is a method, comprising applying a current to an electrode independent of voltage across the circuit that includes the electrode, measuring an electrical property using the electrode, the property existing because of the application of the current to the electrode, and executing voltammetry using the measured electrical property.

In an embodiment, there is a medical device, comprising an implantable portion of the medical device, the implantable portion including at least one electrode, wherein the implantable portion includes a current source, and has as its principle of operation controlling current from the current source to stimulate tissue of a human to evoke a reaction associated with the tissue, and the medical device is configured to control current from the current source to establish a specific electrical phenomenon in reaction to the control of the current source and obtain data based on the electrical phenomenon, which electrical phenomenon and data is sufficient to execute voltammetry.

In an embodiment, there is a cochlear implant, comprising an implantable portion including a receiver-stimulator, the receiver-stimulator including an RF induction coil in signal communication to electronics of the implantable portion, wherein the electronics include a current source, and wherein the implantable portion includes at least a stimulation electrode and a return electrode, wherein the implantable portion is configured to, while implanted in a human, obtain data indicative of a state of a metal-electrolyte interface of the stimulation electrode by varying current of the current source to vary a voltage between the stimulation electrode and a reference.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIGS. 1B-1D are quasi functional diagrams of an exemplary device to which some embodiments may be applicable;

FIGS. 1E and 2A and 2B and 2C and 1F present some schematics related to base technologies associated with some embodiments;

FIGS. 3 and 4 show other exemplary medical devices to which at least some of the teachings herein are applicable;

FIG. 5 shows an exemplary current vs. voltage plot;

FIG. 6 shows an exemplary voltage vs. time plot;

FIGS. 7-9 show exemplary flowcharts for exemplary methods;

FIG. 10 shows an exemplary current vs. voltage plot;

FIGS. 11-13 show an exemplary medical device and features thereof; and

FIG. 14 presents an exemplary diagram of some electrode arrays with some conceptual concepts presented therewith.

### DETAILED DESCRIPTION

Merely for ease of description, the techniques presented herein are primarily described herein with reference to an illustrative medical device, namely a hearing prosthesis. First introduced is a cochlear implant. The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with, at least in conjunction with, another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, retinal implants, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

Note also embodiments include the application of the teachings herein to a medical device that is a non-implanted medical device, such as a minimally invasive probe used by medical personnel.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

The techniques presented herein are also described with reference by way of background to another illustrative medical device, namely a retinal implant. As noted above, the techniques presented herein are also applicable to the technology of vestibular devices (e.g., vestibular implants), visual devices (i.e., bionic eyes), as well as sensors, pacemakers, drug delivery systems, defibrillators, functional electrical stimulation devices, catheters, seizure devices (e.g., devices for monitoring and/or treating epileptic events), sleep apnea devices, electroporation, etc.

Any reference to one of the above-noted sensory prostheses corresponds to an alternate disclosure using one of the other above-noted sensory prostheses unless otherwise noted providing that the art enables such.

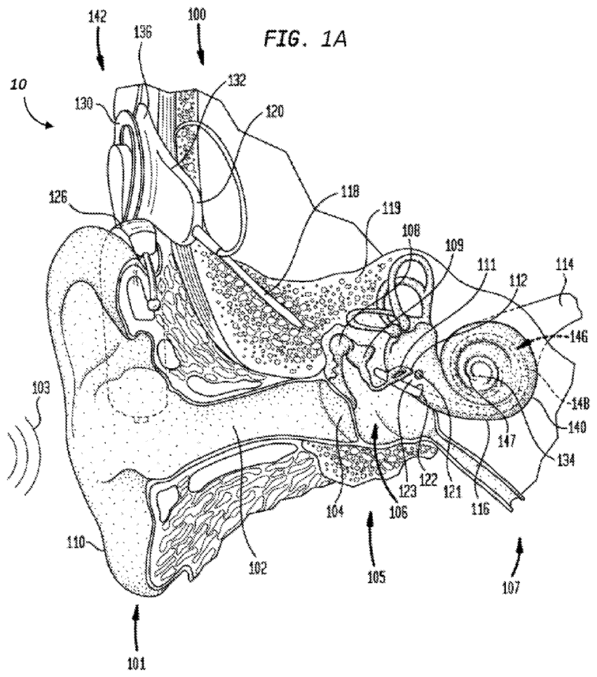

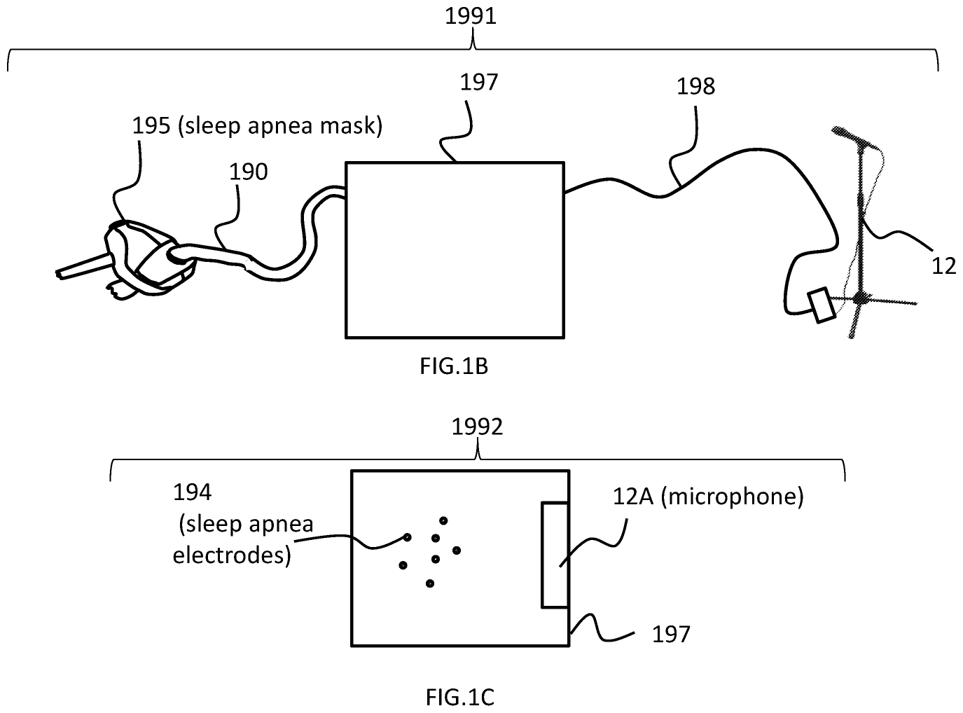

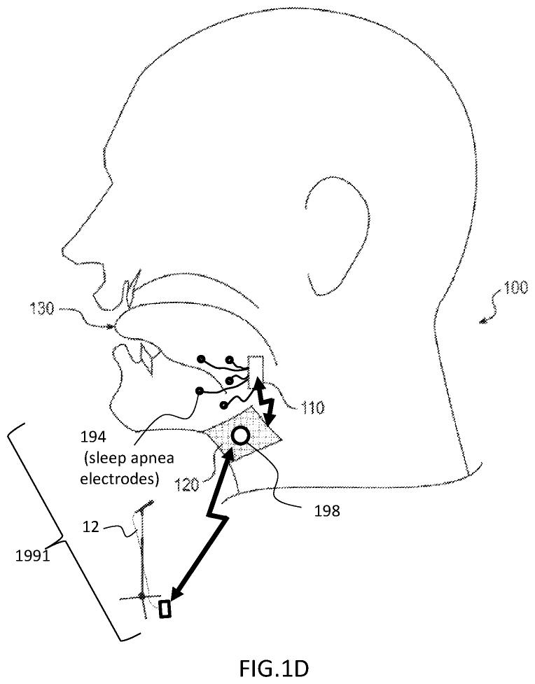

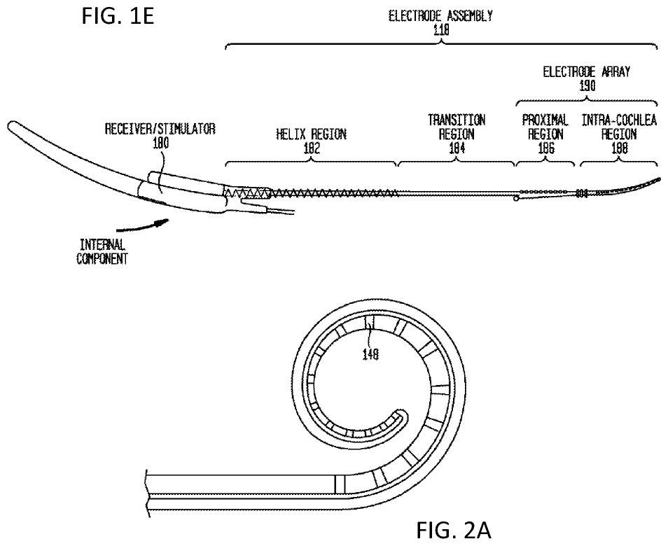

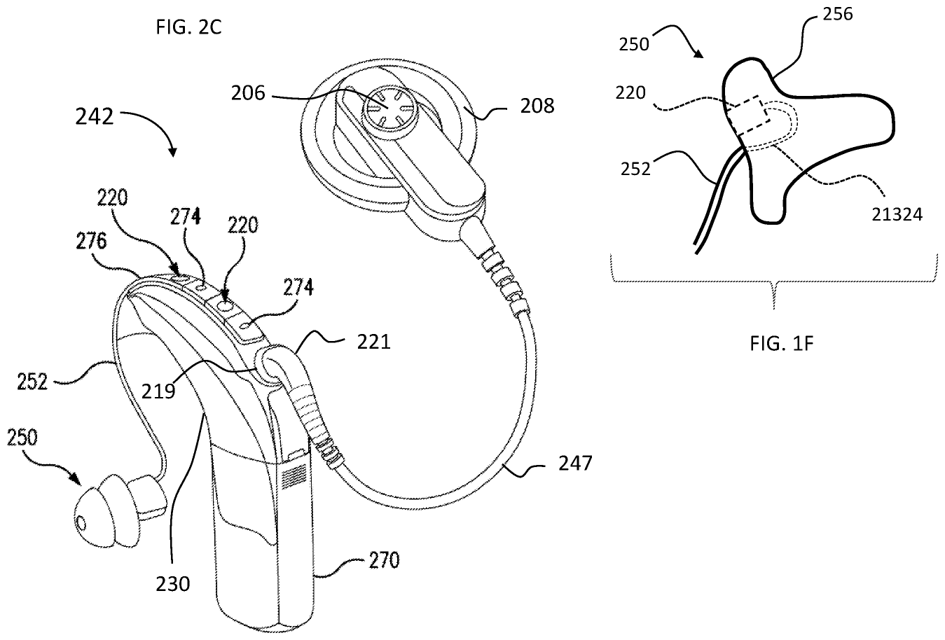

FIG. 1A is perspective view of a partially implantable cochlear implant, referred to as cochlear implant 100, implanted in a recipient. The cochlear implant 100 is part of a system 10 that can include external component(s), as will be detailed below.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive, and inductive transfer, may be used to transfer the power and/or data from external device 142 to cochlear implant 100. In the illustrative embodiments of FIG. 1A, the external energy transfer assembly comprises an external coil 130 that forms part of an inductive radio communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which, in an exemplary embodiment, are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of an electrode assembly 118 utilizing electrodes as elements 148. It is noted that alternate embodiments can utilize other types of stimulating devices. Any device, system, or method of stimulating the cochlea via a device that is located in the cochlea can be utilized in at least some embodiments. In this regard, any implantable array that stimulates tissue, such as a retinal implant array, or a spinal array, or a pacemaker array, etc., is encompassed within the teachings herein unless otherwise noted.

As noted, cochlear implant 100 comprises a partially implantable prosthesis, as contrasted to a totally implantable prosthesis that is capable of operating, at least for a period of time, without the need for external device 142. Therefore, cochlear implant 100 does not comprise a rechargeable power source that stores power received from external device 142, as contrasted to an embodiment where there is an implantable rechargeable power source (e.g., a rechargeable battery). During operation of cochlear implant 100, the power is transferred from the external component to the implanted component via the link, and distributed to the various other implanted components as needed.

It is noted that the teachings detailed herein and/or variations thereof can be utilized with a totally implantable prosthesis. That is, in an alternate embodiment of the cochlear implants or other hearing prostheses detailed herein, the prostheses are totally implantable prostheses, such as where there is an implanted microphone and sound processor and battery.

FIG. 1B provides a schematic of an exemplary conceptual sleep apnea system 1991. Here, this exemplary sleep apnea system utilizes a microphone 12 (represented conceptually) to capture a person's breathing or otherwise the sounds made by a person while sleeping. The microphone transduces the captured sound into an electrical signal which is provided via electrical leads 198 to the main unit 197, which includes a processor unit that can evaluate the signal from leads 198 or, in another arrangement, unit 197 is configured to provide that signal to a remote processing location via the Internet or the like, where the signal was evaluated. Upon an evaluation that an action should be taken or otherwise can be utilitarian taken by the sleep apnea system 1991, the unit 197 activates to implement sleep apnea countermeasures, which countermeasures are conducted by a hose 1902 sleep apnea mask 195. By way of example only and not by way of limitation, pressure variations can be used to treat the sleep apnea upon an indication of such an occurrence.

In an exemplary embodiment, the advanced implantation methods and devices detailed herein can be utilized to treat sleep apnea/in a device that can be used to treat. Specifically, the electrodes of the implant disclosed below can be utilized in place of the electrodes 194 (placed accordingly, of course), and the implant can be of a configuration to treat sleep apnea. In this regard, in an exemplary embodiment, the implantable components detailed herein can be located at locations to treat sleep apnea in accordance with the teachings herein, with the requisite modification if necessary or otherwise utilitarian to implement such.

FIGS. 1C and ID provide another exemplary schematic of another exemplary conceptual sleep apnea system 1992. Here, the sleep apnea system is different from that of FIG. 1B in that electrodes 194 (which can be implanted in some embodiments) are utilized to provide stimulation to the human who is experiencing a sleep apnea scenario. FIG. 1C illustrates an external unit, and FIG. 1D illustrates the external unit 120 and an implanted unit 110 in signal communication via an inductance coil 707 of the external unit and a corresponding implanted inductance coil (not shown) of the implanted unit, according to which the teachings herein can be applicable. Implanted unit 110, can be configured for implantation in a recipient, in a location that permits it to modulate nerves of the recipient 100 via electrodes 194. In treating sleep apnea, implant unit 110 and/or the electrodes thereof can be located on a genioglossus muscle of a patient. Such a location is suitable for modulation of the hypoglossal nerve, branches of which run inside the genioglossus muscle.

External unit 120 can be configured for location external to a patient, either directly contacting, or close to the skin of the recipient. External unit 120 may be configured to be affixed to the patient, for example, by adhering to the skin of the patient, or through a band or other device configured to hold external unit 120 in place. Adherence to the skin of external unit 120 may occur such that it is in the vicinity of the location of implant unit 110 so that, for example, the external unit 120 can be in signal communication with the implant unit 110 as conceptually shown, which communication can be via an inductive link or an RF link or any link that can enable treatment of sleep apnea using the implant unit and the external unit. External unit 120 can include a processor unit 198 that is configured to control the stimulation executed by the implant unit 110. In this regard, processor unit 198 can be in signal communication with microphone 12, via electrical leads, such as in an arrangement where the external unit 120 is a modularized component, or via a wireless system, such as conceptually represented in FIG. 1D.

A common feature of both of these sleep apnea treatment systems is the utilization of the microphone to capture sound, and the utilization of that captured sound to implement one or more features of the sleep apnea system. In some embodiments, the teachings herein are used with the sleep apnea device just detailed.

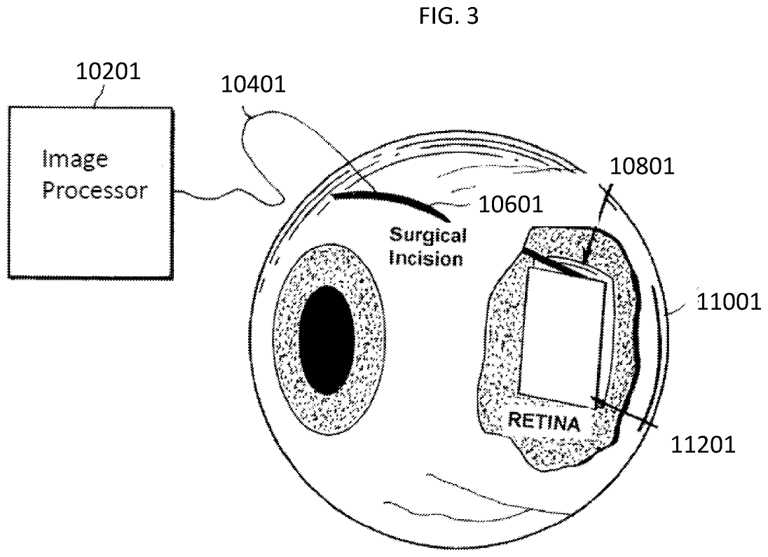

FIG. 3 presents an exemplary embodiment of a neural prosthesis in general, and a retinal prosthesis and an environment of use thereof, in particular, the components of which can be used in whole or in part, with some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

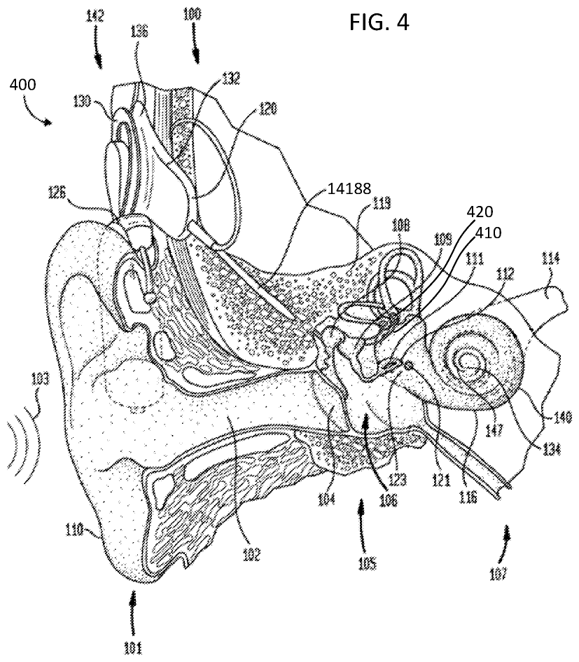

FIG. 4 depicts an exemplary vestibular implant 400 according to one example. Some specific features are described utilizing the above-noted cochlear implant of FIG. 1 in contacts for the various elements. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 400 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith. Any one or more of the teachings herein can be used with the arrangement of FIG. 4.

The implantable component includes a receiver stimulator in a manner concomitant with the above cochlear implant. Here, the vestibular stimulator comprises a main implantable component 120 and an elongate electrode assembly 14188 (where the elongate electrode assembly 14188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 14188.

It is briefly noted that while the embodiment shown in FIG. 4 represents a partially implantable vestibular implant, embodiments can include a totally implantable vestibular implant, such as, where, for example, the motion sensors are located in the implantable portion, in a manner analogous to a cochlear implant.

Elongate electrode assembly 14188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 14188 includes a plurality of leads 410 that branch out away from the main body of the electrode assembly 118 to electrodes 420. Electrodes 420 can be placed at the base of the semicircular ducts as shown in FIG. 4. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

Returning back to hearing prosthesis devices, and in particular a cochlear implant, FIG. 1E is a side view of the internal component of cochlear implant 100 without the other components of system 10 (e.g., the external components). Cochlear implant 100 comprises a receiver/stimulator 180 (combination of main implantable component 120 and internal energy transfer assembly 132) and a stimulating assembly or lead 118. Stimulating assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

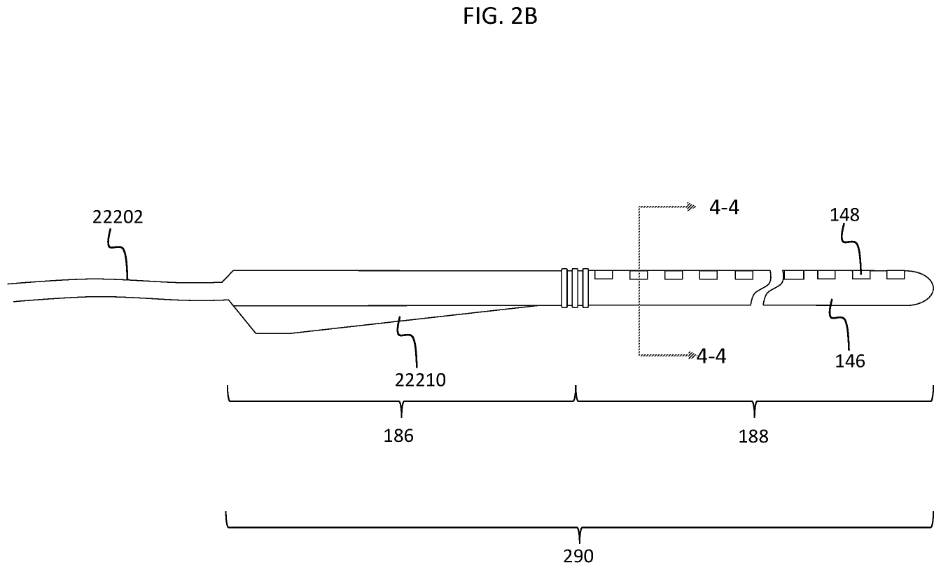

FIG. 2A is a side view of electrode array assembly 190 in a curled orientation, as it would be when inserted in a recipient's cochlea, with electrode contacts 148 located on the inside of the curve. FIG. 2A depicts the electrode array of FIG. 1B in situ in a patient's cochlea 140.

FIG. 2B depicts a side view of a device 290 corresponding to a cochlear implant electrode array assembly that can include some or all of the features of electrode array assembly 190 of FIG. 1B. More specifically, in an exemplary embodiment, stimulating assembly 118 includes electrode array assembly 290 instead of electrode array assembly 190 (i.e., 190 is replaced with 290).

Electrode array assembly 290 includes a cochlear implant electrode array componentry of the 190 assembly above. Note also element 22210, which is a quasi-handle like device utilized with utilitarian value vis-à-vis inserting the 188 section into a cochlea. By way of example only and not by way of limitation, element 22210, which is a silicone body that extends laterally away from the longitudinal axis of the electrode array assembly 290, and has a thickness that is less than that of the main body of the assembly (the portion through which the electrical leads that extend to the electrodes extend to the elongate lead assembly 22202). The thickness combined with the material structure is sufficient so that the handle can be gripped at least by a tweezers or the like during implantation and by application of a force on to the tweezers, the force can be transferred into the electrode array assembly 290 so that section 188 can be inserted into the cochlea.

FIG. 2C presents additional details of an external component assembly 242, corresponding to external component 142 above. It is noted that in a modified form, this device can be used with the other prostheses herein (e.g., some such embodiments might not have the ear piece 250).

External assembly 242 typically comprises a sound transducer 220 for detecting sound, and for generating an electrical audio signal, typically an analog audio signal. In this illustrative arrangement, sound transducer 220 is a microphone. In alternative arrangements, sound transducer 220 can be any device now or later developed that can detect sound and generate electrical signals representative of such sound. An exemplary alternate location of sound transducer 220 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 206 (sometimes herein referred to as a headpiece) comprises an external coil 208 and, a magnet (not shown) secured directly or indirectly to the external coil 208. The signal processing unit processes the output of microphone 220 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals—because element 230 contains this circuitry, the entire component 230 is often called a sound processing unit or a signal processing unit. These coded signals can be referred to herein as a stimulation data signals, which are provided to external transmitter unit 206 via a cable 247. In this exemplary arrangement of FIG. 1D, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 1D that utilizes the bayonet connector.) Conversely, in some embodiments, there is no cable 247. Instead, there is a wireless transmitter and/or transceiver in the housing of component 230 and/or attached to the housing (e.g., a transmitter/transceiver can be attached to the receptacle 219) and the headpiece can include a receiver and/or transceiver, and can be in signal communication with the transmitter/transceiver of/associated with element 230.

FIG. IF provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1F, an ITE component 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). ITE component 250 includes a housing 256, which can be a molding shaped to the recipient. Inside ITE component 250 there is provided a sound transducer 220 that can be located on element 250 so that the natural wonders of the human ear can be utilized to funnel sound in a more natural manner to the sound transducer of the external component. In an exemplary arrangement, sound transducer 242 is in signal communication with remainder of the BTE unit via cable 252, as is schematically depicted in figure IF via the sub cable extending from sound transducer 242 to cable 252. Shown in dashed lines are leads 21324 that extend from transducer 220 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal.

Also, FIG. 2C shows a removable power component 270 (sometimes battery back, or battery for short) directly attached to the base of the body/spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

With the above as a primer (the above should be considered base technologies from which we build upon, and are not part of the invention, but the teachings below can use any one or more of these features in some embodiments, providing that the art enables such), embodiments are directed to cochlear implants and other implants that, in some embodiments, utilize one or more of the teachings above, albeit modified in at least some instances, to practice the teachings herein.

Cochlear implant electrodes are expected to deliver stimulation over the lifetime of the recipient (implantee in this case), potentially, 30, 50, or 75 years or more. Wear of one or more of the electrodes can occur over these time periods (or shorter, in some instances—more on this below), resulting in eventual reduction of utility/reduction of function or loss of utility/function. The smaller the electrode size in general, and the surface area exposed to the ambient environment in particular, the increased rate of passive dissolution/active dissolution, or at least the sooner the electrode will experience of the solution level that deleteriously affects functionality. In at least some exemplary embodiments of the cochlear implant electrode utilized with the teachings detailed herein, an increase in charge density of the electrode results in the increased dissolution rate and/or the shortened utilitarian life expectancy of the electrode/the faster the electrode array reaches the end of its useful life.

And while embodiments depicted above present a 22 electrode cochlear implant electrode array, embodiments include arrays that includes more than 20, 25, 30, 35, 40, 45, 50, 60, 70, 80, 90, 100, 120, 150, 175, 200, 300, 400, 500, 600, or 700 or more electrodes, or any value or range of values therebetween in 1 increments (e.g., 55, 62, 33 to 77, etc.). In an exemplary embodiment, these numbers are located within 1, 1.5, 2, 2.5, or 3 inches, or any value or range of values therebetween in 0.1 inch increments of each other and/or arrayed in a linear or substantially linear fashion that extends the aforementioned distance. In an exemplary embodiment, the aforementioned numbers of electrodes are located within 0.1, 0.2, 0.3, 0.4, 0.5, 0.75, 1, 1.25, 1.5, 1.75, 2, 2.5, 3, 3.5, 4, 4.5, or 5 square inches, or any value or value or range of values therebetween in 0.1 in² increments. Increased electrode density (number of electrodes per a spatial unit) can have utilitarian value with respect to increasing the spectral resolution of a delivered signal (or a sensed phenomenon—as will be detailed below, embodiments detailed herein are not only directed towards devices that provide stimulation to tissue, but are also applicable to devices that sense phenomenon within a human being, such as by way of example only and not by way of limitation, the electrodes of the pacemaker). Thus, embodiments include increasing the spectral resolution of a delivered signal by increasing the number of electrodes within a given spatial dimension. This results in an increased charge density relative to that which would otherwise be the case with a lower number of electrodes in the same given area (where, for example, the working surface of the electrode can thus be larger, because there is more room).

In an embodiment, the surface area of an electrode is 0.1, 0.15, 0.2, 0.25, 0.3, 0.35, 0.4, 0.45, 0.5, 0.55, 0.6, 0.7 0.8, 0.9, 1, 1.25, 1.5, 1.75, 2, 2.25 or 2.5 square mm, or any value or value or range of values therebetween in 0.1 mm² increments.

At least some exemplary embodiments according to the teachings detailed herein apply multipolar stimulation from the electrodes of the implant. This can result in higher charge levels for a given electrode relative to that which would be the case with respect to utilizing those same electrodes for monopolar stimulation. Multipolar stimulation as used in at least some exemplary embodiments herein can have utilitarian value with respect to focusing the stimulation and improving hearing performance, relative to the utilization of monopolar stimulation for example, and can also improve channel independence, and spectral resolution and speech understanding, all relative to that which would be the case in the instance of the utilization of monopolar stimulation. Still, in embodiments that utilize the electrode arrays according to the teachings detailed herein, this can further add to the statistical likelihood and/or actual occurrence of premature electrode dissolution (relative to that which will be the case utilizing monopolar stimulation—all else being equal (all comparisons herein are with regard to all else being equal unless otherwise noted)).

It is noted that in some embodiments, charged balanced biphasic waveforms and use of inert materials such as platinum are utilized to prolong the longevity of the electrodes relative to that which would otherwise be the case.

The rate of dissolution for a particular electrode in at least some exemplary scenarios of the embodiments detailed herein is known, or at least educatedly suspected, to depend on at least one or more factors, such as by way of example only and not by way of limitation, stimulation waveform amplitude and/or pulse width, the number of pulses delivered per hour/day/week, month, etc., position on the carrier of the electrodes, position on the array, position in the body (e.g., position in the cochlea), degree of fibrous tissue growth (relative to zero fibrous tissue growth) and/or chemical composition of the ambient environment of the electrode (e.g., in case of a cochlear implant electrode array, the perilymph inside the cochlea).

In models to which the teachings detailed herein can be applicable, 45% of the variance of the rate of dissolution is not accounted for by charge. Thus, a simple apriori prediction for each individual person or electrode is not feasible in at least some exemplary scenarios of use of the various electrodes/medical devices disclosed herein and other applicable devices. Therefore, whatever measures that are included in a system design to maximize the desired lifetime of the implant, there will remain distribution of dissolution rates that can only be poorly estimated at the time of development/implantation. There will typically be, in at least some exemplary scenarios, a residual risk for each individual recipient that their respective particular dissolution rate will be at the high end of the distribution (or otherwise at a higher end relative to a mean/median and/or mode, for example), resulting in unexpected/unpredicted and premature wear out of their particular implant, potentially necessitating replacement.

It is briefly noted that the phrases “dissolution,” “erosion” and “wear” will be variously used herein to describe phenomenon associated with electrodes. These refer to a change in the state of the electrode itself relative to that which was the case when the electrode was new. This as distinguished from, for example, a film or the like that forms on the electrode surface. That does not change the state of the electrode itself—that is a change to an environment of the electrode that may or may not impact the performance of the electrode. Passive dissolution as used herein refers to the reduction in the amount of material of the electrode owing to chemical reactions due to the environment without electrical current application. Active dissolution as used herein refers to the reduction in the amount of material of the electrode due to use of the electrode (to provide electrical current to the environment). More specifically, the removal of material from the electrode occurs due to (electro)chemical reactions that occur when an electrode is at rest potential (i.e., not used to pass current to the environment) in an electrolyte. Removal of material also occurs due to electrochemical reactions, often at a faster rate, when an electrode is used to provide electrical current to the environment. It is the same electrochemical process, just modified by the changing potential on the electrode that occurs during electrical stimulation. Thus, passive dissolution corresponds to the reduction of material that occurs electrochemically when an electrode sits at rest in an electrolyte, and active dissolution is the process that occurs to remove electrode material when the electrode is used to pass current.

Wear as used herein covers both of these phenomena (as well as erosion), as well as any other phenomenon that may reduce the amount of material of a given electrode over time. Any disclosure of passive dissolution and/or active dissolution corresponds to a disclosure of wear, where the wear is the combination of the two if it is the “and.” For example, if there is a disclosure of determining a passive dissolution rate and/or (or just “and”) an active dissolution rate, that corresponds to determining a wear rate. That is, this corresponds to the combination of the active and passive dissolution (save for the “or,” which is of course is one or the other, and thus the wear is due to the active dissolution or the passive dissolution).

Sometimes, one of these phrases will be used in the absence of another of these words. Unless otherwise noted, any statement herein that utilizes one of these phrases corresponds to a disclosure where such is the case with respect to another one of these phrases, providing that such is technically correct. This is done in the interest of textual economy. This does not mean that they both mean the same thing. This is only to show that the disclosure of one can correspond to a disclosure of an alternate embodiment of the other even though the specific words are not typed onto the page, again in the interest of textual economy.

Dissolution covers both active and passive dissolution, but it is noted that any disclosure herein of dissolution also corresponds to a disclosure of both of the species separately for the purposes of textual economy. This is also the case with wear—a disclosure of wear corresponds to a disclosure of both species of dissolution.

Some embodiments of the teachings detailed herein can include devices, systems, and/or methods that can enable the detection of wear (passive dissolution and/or active dissolution, etc.) and/or otherwise provide an estimation of the status of one or more implanted electrodes with respect to wear beyond that which would be the case with respect to requiring the explantation of the electrodes. That is, embodiments can provide the detection of wear and/or the estimation of wear or otherwise provide an estimation of the remaining lifetime of one or more electrodes implanted in the human body without having to remove the electrodes and analyze the electrodes or otherwise access the electrodes via a surgical/invasive procedure (beyond that which resulted in the electrodes being implanted, of course). In an embodiment, this is done at least in part based on an estimation or detection of the real surface area of an electrode, estimated/detected using voltammetry, such as cyclic voltammetry. Thus, embodiments include estimating or detecting the real surface area of the electrode, and determining/estimating wear features therefrom. In an embodiment, the real surface area that is estimated/detected, is compared to data relating to a non-worn electrode, for example, and wear features are determined based on the comparison.

Some embodiments of the teachings detailed herein provide the aforementioned detection and/or estimation at a level that is not possible with the current state of technology on Jun. 6, 2022, as would be approved by the FDA in the United States of America on that date, and/or as would be approved by the pertinent regulatory authority in the United Kingdom, the Commonwealth of Australia, New Zealand, Canada, the Republic of France, and/or the Federal Republic of Germany, and/or the People's Republic of China.

Some embodiments of the teachings herein provide for the avoidance of a deleterious wear event of a given electrode (e.g., it no longer can stimulate at a utilitarian level) over the electrode's lifetime or otherwise extend the lifetime of the electrode beyond that which would otherwise be the case. By way of example only and not by way of limitation, by implementing any one or more of the teachings detailed herein, an electrode(s) functional life while implanted can be extended greater than 1.25, 1.5, 1.75, 2, 2.25, 2.5, 2.75, 3, 3.5, 4, 4.5, 5, 5.5, 6, 7, 8, 9, or 10 times or longer, or any amount or range of amounts therebetween in 0.05 increments beyond that which would otherwise be the case in the absence of the implementations of one or more of the teachings detailed herein. Thus, embodiments of the teachings detailed herein provide for the determination of a deleterious electrode wear event in progress or that will occur so that utilitarian actions can be taken to remediate or otherwise avoid that event.

Cyclic Voltammetry (CV) is a measurement method that is a useful tool used in some embodiments herein for a number of chemical measurements that are adapted to provide utilitarian insights into electrode status (e.g., wear status)/environment and otherwise a health of the cochlea. Some such useful tools can include, by way of example only and not by way of limitation, diagnosing the state of the metal-electrolyte interface, where, for example, the real surface area of a platinum electrode can be a utilitarian indicator of its roughness and/or an extent to which it has dissolved/worn, etc. In this regard, the surface area of an electrode would sometimes be considered to be the length by the width of a rectangle that results from “rolling out” the electrode to be roughly planar. The part that one can see when looking down on the electrode. Conversely, the real surface area takes into account the fact that the surface is not smooth. By rough analogy, the surface area of the planet Earth is the surface area of a sphere (taken from the equation—with perhaps some adjustment owing to the fact that the earth is not a perfect sphere), but the real surface area would take into account the fact that there are Rocky Mountains and Himalayan mountains and the Alaska Range and the Ural Mountains and the mountains of the African Rift Valley and the Alps and the Andes, etc. These geographic features result in a real surface area of the earth that is larger than that which would result from the equation for a sphere. So it is also the case for an electrode. Over time, the real surface area of the electrode will change, and this can be indicative of the wear rate of the electrode. Accordingly, embodiments can utilize voltammetry in general, and cyclic voltammetry in particular, to ascertain data associated with the real surface area, which data can be utilized to evaluate one or more of the features detailed herein, and otherwise used as a basis to take one or more the actions detailed herein.

Another useful tool can include detection of other chemicals present in the electrolyte that provide a utilitarian signal on a CV, such as, for example, by-products of infection that may produce chemicals that can be detected by a CV, and, for example, detection of a presence of blood through its chemical signature, and for example, sensing of dopamine and other neurotransmitters in the brain that are normally present in very low concentrations. In some embodiments, there are electrodes dedicated for such sensing during intraoperative insertion, for example to monitor for trace signals of blood or other markers of trauma, where a coating on/over one or more electrodes could be bioresorbable, allowing the electrode to be used for other purposes after the coating is dissolved.

In some embodiments, a voltage over a large range is varied, such as, for example, within the “water window” of the electrode, and thus, by way of example only and not by way of limitation, −600 mV to +800 mV vs. Ag/AgCl. Embodiments can include varying the voltage over a range extending from any range of values (inclusive) between −2,500 mV to +4,000 mV or any value or range of values therebetween in 1 mV increments (e.g., a range from (inclusive) −1,205 mV to +3,333 mV, −1,111 mV to +885 mV, etc.). This can be done over a temporal period such as, for example, a few seconds. In an embodiment, the voltage ramp up and/or the voltage ramp down can be meaningfully executed within less than 2, 2.25, 2.5, 2.75, 3, 3.25, 3.5, 3.75, 4, 4.25, 4.5, 4.75, 5, 5.5, 6, 6.5, 7, 7.5, 8, 8.5, 9. 9.5 or 10 seconds or any value or range of values therebetween in 0.01 second increments. (The ramp up need not be the same amount of time as the ramp down, but can be, and the times can be within 70, 75, 80, 85, 86, 87, 88, 89, 90, 91, 92, 93, 94, 95, 96, 97, 98, 99% of each other (the longer time being the denominator). It is noted that the aforementioned times have been qualified as “meaningful.” In this regard, there can be adjustments of current that produce different voltages that are not meaningful to the voltammetry techniques. These would be excluded in some embodiments vis-à-vis qualifying the ramps.

It is briefly noted that embodiments include repeatedly executing voltammetry. Utilitarian value can be achieved by executing a voltammetry analysis two, three, four, five, six or more times with less than 10, 15, 20, 25, 30, 35, 40, 50, 60, 90, 120, 600 seconds between each analysis. The data from the analyses can be averaged or weighted or excluded based on standard practices.

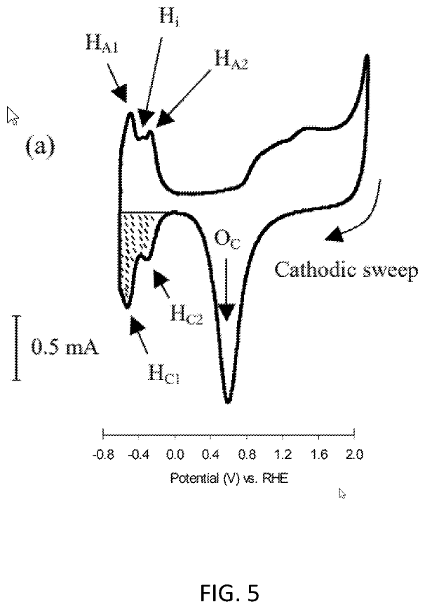

Standard practice in cyclic voltammetry is the variation of potential of the electrode by slowly ramping such up and down while the electrode current is carefully measured. The ramps normally have an upper and lower limit (often set to the water window limits) and are often repeated (cycled) multiple times until stable. An example of this is seen in FIG. 5, for platinum in sulfuric acid showing the shaded area which is proportional to the real surface area of the electrode. From a resulting CV curve for example, which CV curve can be developed using the implants detailed herein for example, data proportional to the real surface area of the electrode can be developed, and from that data, the real surface area can be estimated/extrapolated, and a state of the electrode can be evaluated and then actions can be taken based on that evaluation.

In an exemplary embodiment, because the general shapes of the current versus potential versus RHE or whatever standard is to be used, can be known, the point at which hydrogen ions in the perilymph or otherwise the solution to which the electrode(s) are exposed start plating on the surface of the platinum electrode or whatever material that will make up the electrode will generally be known. In this regard, there will be hydrogen ions in the solution, such as perilymph, which are able to bond with the platinum under the proper circumstances. With reference to FIG. 5, this can be seen with respect to the cathodic sweep at the beginning of the shaded area. Here, as the current increases with decreasing potential, there is an inflection point where the current begins to decrease again around 0V with respect to the curve of FIG. 5. This is where hydrogen begins to bond with the platinum. Consistent with standard practices, current represents a proxy for the number of hydrogen ions that bond to the platinum. Because one platinum atom bonds with one hydrogen ion, the more hydrogen ions that bond to the platinum, the greater the real surface area of the platinum. By way of example, if 10²⁰ hydrogen atoms are deemed to have bonded to the platinum, there will be 10²⁰ platinum atoms that are available for bonding. Thus, the number of hydrogen bonds is a proxy for the real surface area.

As the electrodes dissolve or otherwise degrade over time, the real surface area of the electrode should increase. In an exemplary embodiment, the dissolution is not uniform over the entire surface of the electrode. There will be pockets and craters and “gullies” in areas that experience more dissolution than other areas. Because of this, there will be increases in the real surface area (due to the sides of the craters/pockets/gullies). Thus, there will be an increase in the available platinum on the surface (the real surface) to bond with the hydrogen ions. Because one can deduce the number of hydrogen ions that have bonded to the platinum using the voltammetry curve, one can deduce the real surface area or at least the change in real surface area (the change in the number of hydrogen ions that can bond to the platinum over the pertinent portion of the curve). As noted above, charge is a proxy for the hydrogen atoms that are bonded. By integrating the current over the time, where the x-axis can be the time, the number of hydrogen ions that bond to the platinum can be deduced. That is, the amount of hydrogen plating can be estimated, and thus aspects of the real surface area can be extrapolated from this plating, which can provide a dissolution rate or dissolution state etc. of the electrode. Embodiments can utilize this technique to evaluate the dissolution state or rate or status of an electrode in vivo. These techniques can be executed while the patient is alive and while the electrode is in the cochlea.

In an exemplary embodiment, the voltammetry techniques herein can be executed every time or every other time that the recipient visits a clinician for a fitting checkup or otherwise has a fitting adjustment. That said, embodiments can implement this when recipient is not at an audiologist or otherwise not experiencing a fitting checkup. This can be done automatically or periodically, whether upon instigation by a healthcare professional or by programming of the cochlear implant. In any event, over a number of data points spanning months and/or years and or any of the time frames detailed herein, data sets can be developed that will enable the comparison of the numbers of hydrogen ions that are plating on the electrodes (data for each electrode can be developed, or some electrodes can be proxies for all electrodes, etc.) over the time period, and this comparison will enable an evaluation of the dissolution state of the electrode. (Some embodiments are such that the implant itself, or a remote device such as an app on a smart phone in signal communication with the cochlear implant, can evaluate this data. Other embodiments have the data sent to a professional to have such evaluated (whether by a human or by a computer).

Typically, a baseline data set will be developed at or shortly after implantation. The idea being is that within days or weeks or a month or so of the initial implantation, little to no electrode dissolution will take place. This can be especially the case if the electrodes have not been utilized to stimulate hearing, which would be the case in scenarios where, for example, so-called “turn on” or first activation of the electrode array does not take place for two weeks or a month or so after implantation. In any event, relatively shortly after implantation, one or more data sets are developed for one or more or all of the electrodes that indicate the respective numbers of hydrogen ions that are plating on the electrode utilizing the voltammetry techniques detailed herein (or simply the current vs. time data—it is not necessary to “count” the hydrogen ions—the proxy for the hydrogen ions from the CV data can be sufficient). If these techniques are repeated every six months for example, trendlines can be developed and/or a comparison can be constantly made to the initial data set. If the percentage increase in the number of hydrogen ions relative to the initial data set increases above a certain threshold, it can be determined that there is dissolution that might have a deleterious impact occurring. Any of the mitigation and/or treatment techniques detailed herein can be applied. The point is that utilizing the data from the voltammetry techniques detailed herein can enable the real surface area to be evaluated so that dissolution rates can be determined and/or dissolution states can be determined and from that, actions can be taken in accordance with any of the teachings detailed herein.

Heretofore, it has been standard practice that CV is implemented via the generation of a very slowly ramping voltage waveform (over a voltage range of up to about 1400 mV by way of example only, over a period of seconds) and the measurement of current. That is, voltage is controlled. This is implemented with a voltage source (as opposed to a current source). This generation of a voltage coupled with measurement of a current does not mesh with standard cochlear implant systems, as cochlear implant systems, at least those approved in one or more of the jurisdictions noted above are designed to do the opposite: generate a current using a current source, and measure a voltage. Embodiments thus include devices, systems and/or methods that exclude the generation of a voltage and measure current based on the generation of the voltage.

Embodiments can provide a control system that can approximate the ideal CV system (that established by varying voltage and measuring current) using the existing hardware already available in approved cochlear implants that are licensed for use/approved for use, as of Jun. 6, 2022, in any one or more or all of the jurisdictions noted above, such as for example, FDA approved cochlear implants in the United States. Embodiments can include a cochlear implant that can make multiple rapid measurements of the electrode voltage and use a control system to vary the current source of the cochlear implant generate an approximation to a constantly ramping voltage, as required by the CV measurement. The measurement of current can be executed by reading the value of the current source. In this manner, the implant does not need extra hardware to generate the voltage and/or to measure current. Thus, the cochlear implant is used as current source and a voltage reader, as opposed to that which is the case in traditional CV, where a voltage source and a current reader is used.

Embodiments can thus include control system that can approximate the ideal cyclic voltammetry (CV) system using the existing hardware already available in a cochlear implant. In some of these embodiments, the existing hardware includes a current source rather than a voltage source for stimulating a cochlea. The implant can make multiple rapid measurements of the electrode voltage and use the control system to vary the current source output to generate an approximation to a ramping voltage, such as a constantly ramping voltage, as is required in CV measurements. The measurement of current can correspond to reading the value of the current source output. No extra hardware is used in some embodiments to generate the voltage or measure current.

Some embodiments enable the generation of a sufficiently accurate approximation of the voltage ramp in this manner because of the relatively slow speed of the voltage ramp and the long measurement times, coupled with the ability of cochlear implant current sources to generate rapidly varying current values and the ability of cochlear implant voltage measurement circuit to make high speed voltage measurements.

Some exemplary embodiments are described below, often in terms of a cochlear implant. However, it is noted that in addition to cochlear implants, the teachings herein can have applicability to other types of implants, including, for example: deep brain stimulators, pacemaker, bladder stimulation, and drug delivery implants, including pump (e.g., insulin pump) implants as well as non-pump implant (e.g., non-pump drug-eluting implant). Corollary to this is that the teachings herein can be used in non-implant/prosthetic implementations, and in non-medical device applications (e.g., the techniques herein can be used for CV where a current source is available instead of the traditional voltage source).

Moreover, any one or more or all of the teachings herein can be used in other types of voltammetry other than cyclic voltammetry. Embodiments can include a device for and a method of using voltammetry to detect the concentration of a drug in an electrolyte (e.g., cochlear fluid/perilymph). Embodiments can use this detection as a basis to regulate the delivery of the drug (automatically and/or in a prescriptive manner). Additional details of this will be described below.

It is noted that while embodiments focus on the use of a device that has a current source, embodiment can include using a device that is configured to vary a voltage source thereof to vary a voltage applied to the at least one electrode to practice voltammetry. Embodiments thus include adding a voltage source device (adding hardware) to an existing cochlear implant design (or developing a new design) in addition to having a current source, where the voltage source is utilized to implement voltammetry (the current source is idled or disconnected temporarily). After using the voltage source, the voltage source can be idled or disconnected from the power source and the current source can be used to implement electric hearing. Thus embodiments include engagement of at least one electrode by maintaining a fixed voltage independent of a load resistance and/or an output current.

Embodiments can include a medical device, such as a cochlear implant, comprising an implantable portion of the medical device. In this exemplary embodiment, the implantable portion of the medical device and include at least one electrode, such as one of the electrodes of the electrode array or the return electrode, which can be located outside the cochlea on a separate lead separate from the lead that supports the electrode array, or an extra cochlear electrode mounted on the leads of the electrode array (but positioned so it is outside the cochlea), all by way of example, and/or an electrode that is utilized as the return electrode that is located on the housing of the receiver stimulator of the cochlear implant. In this embodiment, the implantable portion is configured to, while implanted in a human, obtain data indicative of a state of a metal-electrolyte interface of at least one electrode.

In an embodiment, the data indicative of a state of the metal-electrolyte can be latent variables for example that can be utilized to ascertain or otherwise estimate a passive dissolution state and/or active dissolution state of an electrode (and thus a wear state of the electrode). In an exemplary embodiment, features associated with voltammetry in general, and CV in particular, can be impacted by properties of an electrode and/or properties on a surface of the electrode at different points during its lifetime. These properties can be measured by executing one or more of the CV teachings herein with a cochlear implant. This can be executed by passing a measurement current between two electrodes, such as, for example, electrode 6 out of the 22 electrodes of an electrode array and an extra cochlear electrode. As will be detailed below, additional electrodes can be used. Any method of measuring and/or obtaining electrical properties that can enable the teachings herein can be used in some embodiments, providing that such is safe for the recipient, unless otherwise indicated.

The properties that are measured that can enable the ascertention of wear can be measured by implementing the teachings and/or modified teachings of the patent applications detailed below by Carter and/or Pawsey and/or Melman, albeit in a controlled manner different from what is taught by those patent applications. Accordingly, in an exemplary embodiment, there is an implantable portion of a medical device that includes an electrode array, such as a cochlear implant, or any other device that utilizes electrodes such as those detailed herein or others to which the teachings herein are applicable, where the array comprises a plurality of electrodes, and a carrier carrying the electrodes. In this embodiment the implantable medical device is configured to enable in vivo analysis of a state of a metal-electrolyte interface of at least one electrode.

Referring back to the example where the medical device is configured to obtain data indicative of a state of the metal-electrolyte interface, in an exemplary embodiment, the obtained data is based on a chemical reaction that takes place owing to energizement of the at least one electrode independent of voltage across the circuit that includes the electrode. This as opposed to what would be the case if a voltage source was being used to energize the electrode.

In some embodiments, the obtained data is data impacted by a real surface area of the electrode. In this regard, as noted above, the real surface area of a platinum electrode can be a good indicator of its roughness. Corollary to this is that an indication of its roughness, or more accurately, latent variables which are related to the electrodes roughness, can be used to determine electrode wear, or at least estimate electrode wear (whether passive and/or active).

Concomitant with the use of a no-new hardware cochlear implant (as differentiated from programming/modifications to firmware—embodiments include taking current cochlear implants and updating/changing the software and/or firmware to implement the teachings herein—a firmware update can be used, which updated firmware can drive existing hardware to implement the teachings herein—embodiments include updating existing cochlear implants implanted/provided to a recipient that did not have the voltammetry abilities disclosed herein to have such), the medical device is configured to obtain the data by generating a current and measuring voltage. Thus, the medical device can be a device, such as a cochlear implant, that includes no extra hardware to generate a voltage or measure current than that which is used during the normal function of the device (e.g., cochlear implant) to operate per its traditional function (e.g., to evoke a hearing percept). In an exemplary embodiment, the medical device uses the exact same hardware that is utilized to operate according to its normal/traditional function that utilizes to obtain the data indicative of a state of a metal-electrolyte interface, or otherwise to generate a voltage and/or to measure current. In this regard, in an exemplary embodiment, the medical device utilizes only new firmware and/or software to implement the action of obtaining the data. In an embodiment, the components that create the current source are maintained/not changed, but the components that control the current output are changed/modified. In an embodiment, the capacitors, resistors and/or transistors that are used to implement the current source for the cochlear implant are kept the same. The control is what is different.

Corollary to the above is that the aforementioned restrictions could be directed to the implant as opposed to the entire medical device. By way of example, the implant could have no new hardware. Moreover, the implant could be controlled from an external device for another device to implement the actions of obtaining the data. Indeed, in some embodiments, the implant (e.g., implant 100 of FIG. 1A) includes no new programming and/or new firmware relative to devices that were traditionally used for otherwise use prior to the implementation of the teachings detailed herein, such as devices approved for use in any one or more of the aforementioned jurisdictions. In this regard, an exemplary method includes controlling, utilizing the external device or utilizing a device that is configured to communicate with the implantable portion of the cochlear implant, the implantable portion of the cochlear implant to implement any one or more of the actions detailed herein, where the implantable portion is completely agnostic as to how it is controlled. Indeed, in an exemplary embodiment includes utilizing an implantable portion of a cochlear implant the exact design of which is in production/is approved for use as of Jun. 6, 2022, or a comparable design, and to execute any one or more of the actions detailed herein using such by controlling the implantable portion utilizing an external device or another device in communication there with. That is, in an exemplary embodiment, the coded signals that are transcutaneously transmitted (or transmitted non-transcutaneously, such as can be the case with respect to an embodiment where the features associated with the metal surface are being monitored during implantation or prior to the end of surgery, where, for example, an external device such as that shown in FIG. 1A or a special purpose lab device is utilized to transmit the coded signals to the implantable portion) are utilized to control the implantable portion two very the current according to the teachings detailed herein, or otherwise control the current generator of the implantable portion, directly or indirectly, so that voltammetry in general, and/or cyclic voltammetry specifically, can be executed.

In this regard, in an exemplary embodiment, the implantable component includes a current source that is powered from inductance generated current in the receiver coil owing to the induction field generated by the external component or otherwise the surgical component that is utilized to power the implantable portion of the cochlear implant (in the case where the implant has no long term power source, such as a batter (as opposed to capacitors)). Any device system and/or method that can take a direct current and/or alternating current and provide a current controlled output can be utilized in at least some exemplary embodiments. In an exemplary embodiment, the current source is a compilation of one or more of transistors, resistors and/or capacitors and/or other electrical components that provide electricity to the electrodes in the form of a current source or otherwise function as a current source. In an embodiment, no new capacitors and/or transistors and/or resistors are added to existing designs of implants approved for use/used per the temporal periods herein and/or are modified or removed.

In an exemplary embodiment, in the case of a cochlear implant, the components that are utilized to provide current to the electrodes to implement the voltammetry according to the teachings detailed herein are the same components that are utilized to provide current to the electrodes to evoke a hearing percept. In an exemplary embodiment, it is the control of this current that is adjusted relative to that which is the case when implementing the hearing function of the cochlear implant that is the difference between the cochlear implant according to the teachings detailed herein and prior implementations of cochlear implants.

In an exemplary embodiment, the components that are utilized to implement the voltammetry according to the teachings detailed herein can be used to evoke a hearing percept within 60, 30, 15, 10, 9, 8, 7, 6, 5, 4, 3, 2, 1, 0.5 or 0.25 minutes or less from when these components were last utilized to implement voltammetry and/or visa-versa. In an exemplary scenario of use, during a first temporal period, components of the implantable portion are utilized to evoke a hearing percept in the ordinary and customary manner that is the case for a cochlear implant. Those same components that were utilized to evoke a hearing percept (the components of the implant) vis-à-vis current generation are then used to implement voltammetry during a second temporal period following the first temporal period. Then, during a third temporal period after the second temporal period, those components and again utilized to evoke a hearing percept in the ordinary and customary manner. That said, in an exemplary embodiment, the first time that the electrodes are used can be for voltammetry. Indeed, as noted above and as will be described in greater detail below, voltammetry can be executed upon implantation of the electrode array in the cochlea prior to the completion of the surgical procedure/while the recipient is in the surgery operating room. Corollary to this is that in some embodiments, the voltammetry can be utilized before first activation of the cochlear implant and/or otherwise before evoking a first hearing percept with the cochlear implant. Embodiments can include doing both: utilizing the current source to implement voltammetry prior to first activation, and then periodically under randomly utilizing the current source for voltammetry after the implant has been utilized to evoke a hearing percept for testing purposes or otherwise for monitoring purposes during the lifetime of implantation of the cochlear implant. In any event, the aforementioned temporal periods can directly follow one another. The point is that in at least some exemplary embodiments, it is only a matter of controlling the same components differently when implementing voltammetry relative to the normal functionality of the cochlear implant which is to evoke a hearing percept. The same current source that is utilized to stimulate tissue via the electrodes can be controlled in a different manner to implement voltammetry. This as opposed to physically altering or otherwise adding componentry to the cochlear implant (at least the implantable portion) to implement voltammetry.

As will be described in greater detail below, in at least some exemplary embodiments, the provision of current to the electrode is simply a matter of controlling the current applied to the electrode in a different manner than that which would be the case to evoke a hearing percept.

Of course, the implementation of voltammetry includes the ability to read potentials or otherwise measure potentials between the electrode of the electrode array and the return electrode and/or another electrode. In this regard, most standard cochlear implants are configured to read voltages. Thus, reading the voltages is a matter of utilizing existing components in the cochlear implant, but now for purposes of voltammetry.

In view of the above, it can be seen that in some embodiments, the implant is an implant that has a current source that is used to energize the electrodes, such as the tissue stimulating electrodes in general, and the electrodes of the cochlear electrode array in particular. In some embodiments, there is no voltage source associated with energizing the electrodes. The medical device is configured to vary a current source thereof to vary a current applied to the at least one electrode, and the data that is obtained that is indicative of a state of a metal-electrolyte interface is voltammetry data, and the data can be cyclic voltammetry data.

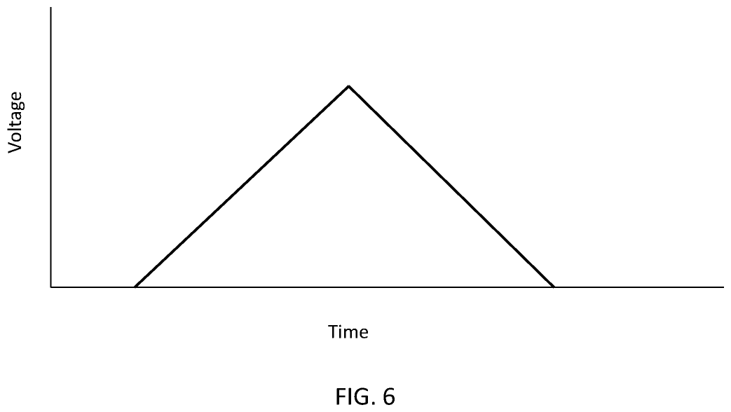

Embodiments thus include a medical device that includes a control system that varies the current applied to the electrode to generate an approximation to a ramping voltage in general, and a constantly ramping voltage in particular. FIG. 6 shows an exemplary voltage ramp relative to time that can be implemented, or more accurately, that is approximated, in an exemplary embodiment. As seen, in this exemplary embodiment, the ramp up is linear as well as the ramp down. And it is briefly noted that the zero point for the Y axis does not necessarily equate to zero voltage. Indeed, embodiments include starting of the voltage at a negative value and then ramping up to zero or some other value and then continuously ramping up to a positive value, before ramping down again. It is further noted that the path to get to the starting voltage need not be a ramp or otherwise need not be linear. The disclosed ramping is presented for the scenarios where the implant is utilized for voltammetry, or, more accurately, during the period where current is applied for voltammetry.

Features of how the current source is used to approximate the ramping voltage will be described below.

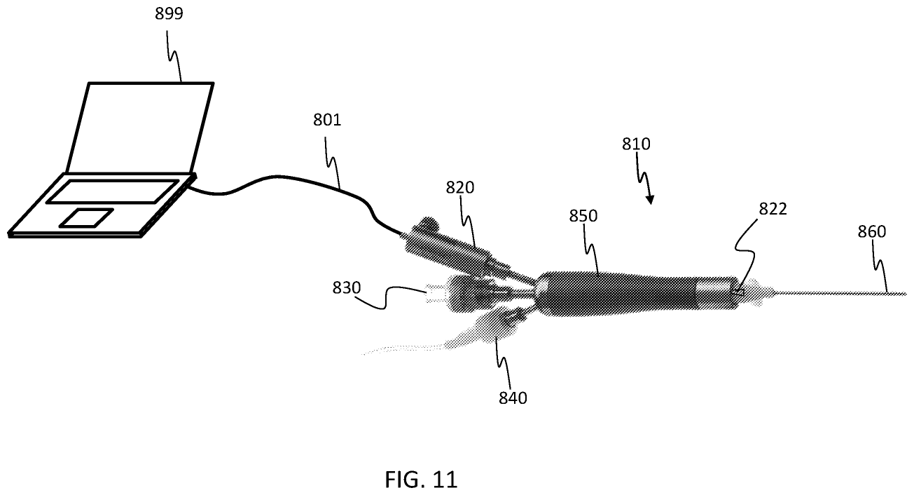

In an embodiment, there is a medical device, comprising at least one electrode and electronics configured to controllably apply an electrical signal to the electrode, wherein the medical device is configured to enable in vivo obtention of data indicative of a chemical in an environment of the at least one electrode via voltammetry. While embodiments herein are described in terms of an implantable device, such as a cochlear implant, it is noted that the teachings herein are also applicable to non-implant medical device. Embodiments can include medical devices used for surgery and/or for postoperative diagnosis. Indeed, embodiments can include devices that are unrelated to surgery. Embodiments can include devices that are utilized for minimally invasive testing before surgery or even for humans who will not have the surgery or have not had a surgery related to the testing. FIG. 11 shows a medical device that is a non-implant medical device. In this embodiment, this is an ear system endoscope 810 that includes various components, such as, an optical channel 820 which enables data indicative of an image inside the ear system to be conveyed to a location outside the ear system. This can be based on fiber optics or wired communication. (While this embodiment is described in terms of an ear system endoscope, other embodiments include other medical devices utilized for other body parts, such as a heart cavity or a long, or the cavity in which is located the brain by way of example. This can also be utilized for an eyeball were for a bladder or kidney or a fetal sack or any portion inside the body to which the teachings can be applicable, of the requisite modifications to access such.) Corollary to this is that there is a camera 822 or some other light capture arrangement (a purely optical device can be used, which may magnify light captured at the working end) that is part of the ear system endoscope. Data from the handheld device can be communicated to a laptop 899 or a desktop computer, which could be located in the same room in which the ear system endoscope is being utilized to reach the cochlea.

The ear system endoscope 810 further includes a surgical tool port 830. This port can be configured to receive a needle, such as needle 2260 described below, which includes electrodes for voltammetry) and/or a drill bit (which can also include electrodes—the drill can have a hollow lumen—the drill bit can be used to drill through bone or hard tissue an access an area of the body behind such). Furthermore, in an example, the port can receive a biopsy tool and/or blades and/or forceps and/or microneedles (microneedle array/assembly), catheters, etc. Moreover, by way of example only and not by way of limitation, in an exemplary embodiment, tools that are configured to take a sample of a fluid or a liquid within the body, such as perilymph within the cochlea and/or the fluid within the semicircular canals, example, can be extended through the aforementioned port(s).

An exemplary embodiment includes a steerable tip endoscope with a channel for vision and a channel for tools (e.g., a drill tip and/or a therapeutic substance delivery tool (e.g., a conduit extends through a channel), the tool bending in compliance with the tip angle. Thus, there could be 2, 3, 4 or more surgical tool ports 830. The needle 2260 can be a steerable needle.

Also, as can be seen, there is an irrigation port 840, which can be utilized to provide irrigation fluid, such as a saline liquid, to the working end of the ear system endoscope, which can be used to provide irrigation of the middle and/or inner ear during use of the tool. The tympanic membrane can also be irrigated utilizing the irrigation features of the ear system endoscope in some embodiments.

As can be seen, the channel 820 and the ports 830 and 840 are supported by a body 850, which can be ergonomically designed so that a surgeon or other healthcare professional and easily grip and support the ear system endoscope with a thumb and one or more fingers of a hand, or the entire hand.

The working end of the ear system endoscope 810 includes a termination 860, which can be a tube made of metal, such as stainless steel (e.g., 316), or some other material. In an exemplary embodiment, the termination 860 can correspond to those of at least the body portion (which may or may not include the sharp end) of a syringe termination approved for use in the United States as of Jun. 6, 2022.

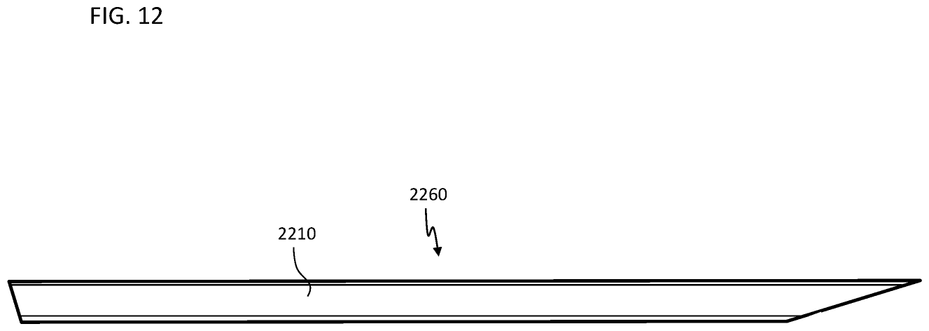

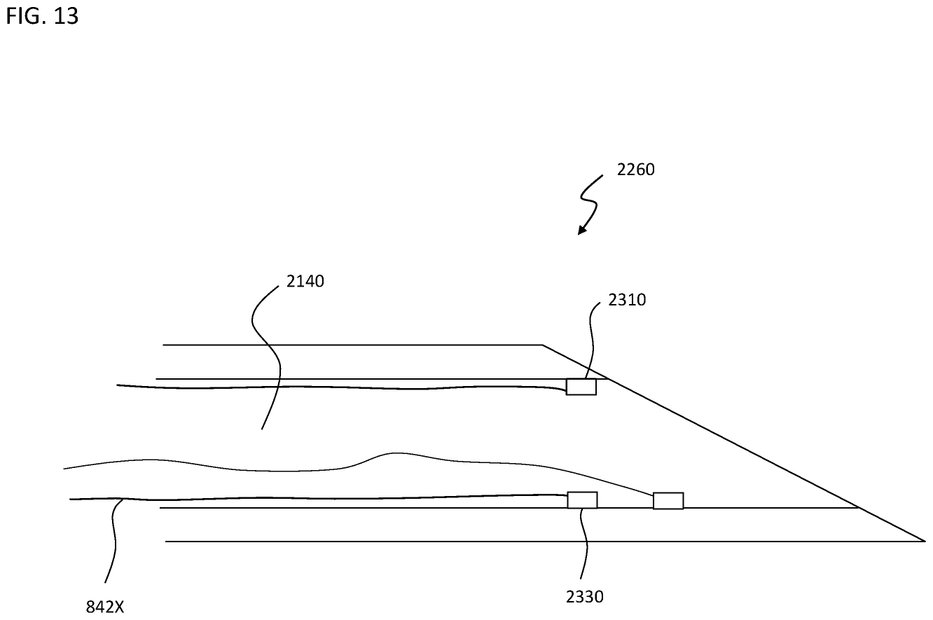

In an embodiment, the termination 860 can be used to guide a probe into the cochlea (e.g., through the round window for example), which probe can have the aforementioned electrode. FIG. 12 presents an exemplary termination 2260/needle 2260 that includes a lumen 2210 configured to move through the termination 860 and puncture tissue of a human to reach a space in a human in which an electrolyte or some other body fluid is located. Inside the lumen, as seen in FIG. 13, are electrodes 2310 and 2330, one of which can correspond to the aforementioned at least one electrode (note that such devices can include any number of electrodes that can enable the teachings herein, such as any number of electrodes as detailed herein (e.g., 22 electrodes for example). In this embodiment, electrode 2310 can be an electrode that has a coating or is made of a material that is conducive to executing voltammetry to detect a drug for example, and electrode 2330 can be made of a material and/or has a coating that is conducive to executing voltammetry to detect chemicals that are a byproduct of infection, and electrode 2333 that is made of a material and/or has a coating that is conducive to executing voltammetry to detect chemicals that are indicative of blood in the cochlea, for example.

These electrodes are connected to electronics to implement voltammetry via leads 842X. And note that the return electrode is not shown. This can be located on the termination 860, or on the termination 2260, or elsewhere.

In use, the needle 2260 is inserted into a body space, such as a cochlea, and body fluids, such as fluids that contain or otherwise are electrolytes, come into contact with one or more of the electrodes. Voltammetry is thus executed in a manner concomitant with the other teachings detailed herein. The difference between this embodiment and the embodiments where the electrodes are part of an implant is that the electrodes are removed from the human in short order after the voltammetry is executed,

Any disclosure associated with an implant corresponds to a disclosure of a non-implant device, and visa-versa, unless noted otherwise and unless the art does not enable such.

In an embodiment, again referring to a medical device, such as a cochlear implant that has a plurality of electrodes and a carrier carrying the plurality of electrodes, and that has electronics configured to controllably apply an electrical signal to the electrodes of the plurality of electrodes to stimulate tissue of a human, the medical device is configured to enable in vivo obtention of data indicative of a chemical in an environment of least one electrode of the plurality of electrodes via voltammetry. In an embodiment, this is executed with the electrodes in their normal state as would be used for evoking a hearing percept or otherwise a sensory percept. In another embodiment, this is executed via electrodes that are in a state that is different from the normal operational state. By way of example only and not by way of limitation, the at least one electrode includes a coating that is bioresorbable, which coating influences the voltammetry (e.g., CV) in the presence of certain substances. In some embodiments, the coating is bioresorbable and configured so that, upon bioresorption, the at least one electrode is usable to stimulate the tissue of the recipient (in the normal traditional manner).

In an exemplary embodiment, the coating is dissolved or otherwise be bioresorbed within 30, 25, 20, 15, 10, 5, 4, 3, 2, 1, 0.5, or 0.25 days of implantation into the body, at least by an amount of 70, 75, 80, 85, 90, 95 or 100% or any value or range of values therebetween in 1% increments. In an exemplary embodiment, a subset of the electrodes could have this coating. In an exemplary embodiment, 1 or 2 or three or more channels, or more accurately, electrodes corresponding to specific channels, are provided with a coating. In an exemplary embodiment, an electrode that is deemed to be of lesser importance with respect to evoking a sensory percept can be provided with a coating. By way of example only and not by way of limitation, the more apical electrodes can be provided with the coating. The idea here can be that the cochlear implant can be used as a hearing prosthesis for most all of the frequency ranges, including the lower and mid frequency ranges, prior to the dissolution of the coating on the more apical electrode(s). This will provide the recipient with the utility of the cochlear implant, albeit in a slightly limited manner relative to that which would otherwise be the case. Because a high-frequency sound may not be as important to the recipient to hear as lower frequency sounds, the relatively short period where one or more of the higher frequency channels is not utilize a ball or otherwise is of limited use relative to that which will ultimately be the case is a minor inconvenience relative to the utilitarian value of being able to implement CV with the electrode array in the cochlea.

And while the above noted coatings are presented in terms of being bioresorbable, some embodiments, the coatings need not be bioresorbable, and otherwise can be permanent. In an exemplary embodiment, one or more channels are “sacrificed” for the cause of enabling targeted voltammetry (e.g., targeted to a specific chemical or to a specific phenomenon). By way of example only and not by way of limitation, a 22 electrode array can have 22 channels, one channel for each electrode. One of these electrodes could be selected for modification or otherwise to have the coating. That electrode would not be used during the normal course of the utilization of the cochlear implant, but instead would be utilized to implement voltammetry as desired. That said, in some other embodiments, there can be dedicated electrodes in addition to the normal 22 electrodes. In an exemplary embodiment, electrodes for voltammetry can be located on the opposite side of the carrier from the electrodes. For example, there could be 1, 2, 3, 4, 5, 6, 7, 8, 9, 10, 11, 12, or more electrodes located opposite the electrodes that face the modiolus that are used to evoke a hearing percept during normal operation the cochlear implant. These electrodes could have dedicated leads that run to the electronics package of the receiver-stimulator. These electrodes could be the electrodes that are utilized for CV for example. In an exemplary embodiment, respective electrodes could have one or more specific coatings that react differently to different chemicals or otherwise different environments. Thus, voltammetry could be practiced to detect different chemicals on different electrodes. In an exemplary embodiment, one or more voltage ramp ups and voltage ramp downs would be respectively implemented on respective electrodes to identify the presence of a respective chemical or a plurality of respective chemicals that are to be detected with a given electrode. Respective electrodes could thus be configured to detect respectively different chemicals. Embodiments include executing voltammetry with one or more or all of the electrodes (temporally separated, e.g., electrode 5 could be used during a first time period, and then electrode 8 could be used during a second time period after the first (separated by seconds or minutes or more), etc.

It is briefly noted that this concept can also be applied to the tissue stimulating electrodes, albeit in a manner where the coating would be bioresorbable. Again, dedicated electrodes to implement voltammetry could have utilitarian value with respect to utilizing coatings or surface treatments that need not be resorbable, and thus can be utilized throughout the life of the cochlear implant, or at least for a relatively long period of time, relative to that which would be the case for the embodiments that utilize bioresorbable coatings.

Thus, embodiments can include a cochlear implant where the hardware is modified relative to that according to the standard implants. But note, at least with respect to the embodiments where the existing electrodes are coated, the hardware associated with the electronics package of the receiver-stimulator could still be unmodified. That is, while the implantable portion is modified in the sense that a coating is applied to the electrode, the underlying electronics package remains unmodified. Indeed, no internal component of the implant is modified. Instead, only the external portion of the implantable portion is modified (with the coating).

Thus, it can be seen that in an exemplary embodiment, the at least one electrode includes a coating that is bioresorbable, which coating influences the voltammetry (e.g., the CV).

In an embodiment, the chemical reaction is a result of at least one of blood in the environment, products produced by infection, inflammation or a neurotransmitter. As will be described below, embodiments include the utilization of voltammetry for example to detect or otherwise identify the likelihood that a given scenario is taking place within the recipient, and based on that, take one or more actions to address this phenomenon. As will be describe below, such actions can include the application of a therapeutic substance, automatically or manually.

In some embodiments, the medical device is configured to identify a current level at which a chemical reaction begins, the current level being the data that is obtained indicative of the chemical in the environment. The current level can be based on known voltammetry characteristics/scenarios, or can be based on empirical data obtained using a given model of cochlear implant in general, and a given model of an electrode array in particular (platinum electrodes). Moreover, there is the underlying issue of what currents will result in the approximate voltage curve that sufficiently corresponds the ideal voltage. In this regard, embodiments can include applying experimental currents for various times to obtain data corresponding to the resulting voltage readings. For example, prior to implementing the voltammetry techniques detailed herein, a set of base data could be obtained so as to get a “feel” for what currents will produce what voltages. Thus, by way of example, test currents will be applied in varying magnitudes and/or varying lengths of time, to obtain test voltages. This test data would then be utilized to develop an embryonic control regime to implement the voltammetry. This data could be refined over various executions of voltammetry and thus the control regimes can be updated. This test data can be for an individual human, and thus the unique to a given human. That said, embodiments can utilize a statistical basis for the initial control regime, which statistical basis can be based on similarly situated humans where voltammetry has been implemented utilizing the techniques detailed herein in the past.

Embodiments can include a device that is configured to vary non-zero current applied to respective electrodes by less than, greater than and/or equal to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 85, 80, 85, 90, 95, 100, 105, 110, 120, 130, 140, 150, 175, 200, 225, 250, 275, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4500, 5000, 5500, 6000, 6500, 7000 or 7500 μA (positive or negative—some embodiments can go from zero to these numbers, and some can go from the negative to the positive in the following timeframes) or more or any value or range of values therebetween in 0.1 μA increments in a time period less than and/or equal to 0.05, 0.1, 0.15, 0.2, 0.25, 0.3, 0.35, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 85, 80, 85, 90, 95, 100, 105, 110, 120, 130, 140, 150, 175, 200, 225, 250, 275, 300, 350, 400, 450, 500, 550, 600, 650, 700, 750, 800, 850, 900, 950 or 1000 μs or any value or range of values therebetween in 0.01 μs. For example, in an embodiment, current source rise and fall time can be less than or equal to 2, 3, 4, 5, 6 or 7 μs, and the current can go from zero to greater than 2 mA or from less than −2 mA to greater than 2 mA (or from less than −1 mA to +2 mA—the range need not be symmetrical). The current can be stepped up (or down) from zero current to full current in a few microseconds. Stimulation rates up to 30k stimulations per second or even higher can be executed in some embodiments (˜30 μs per stimulation).

In an embodiment, each stimulation can be two (2) pulses (one positive, one negative) and there can also be an idle time and/or shorting time. In such an embodiment, the rise and fall times will be relatively short to achieve maximum utilitarian value.

In an embodiment, the current source has a resolution of less than and/or greater than and/or equal to 0.5, 0.6, 0.7, 0.8, 0.9, 1, 2, 3, 4, 5, 6, 7, 8, 9, 10, 11, 12, 13, 14 or 15 microamps or any value or range of values therebetween in 0.05 microamp increments. In some embodiments, the maximum output is less than, greater than and/or equal to 0.01, 0.05, 0.1, 0.15, 0.2, 0.25, 0.3, 0.35, 0.4, 0.45, 0.5, 0.6, 0.7, 0.8, 0.9, 1, 2, 3, 4, 5, 6, 7, 8, 9, 10, 11, 12, 13, 14, 15 milliamps or any value or range of values therebetween in 0.0001 milliamp increments.

For example, in a medical device that is configured to vary non-zero current applied to respective electrodes by over 2 mA per 5 microseconds, if stimulation periods are broken up into 1000 cycles per second, the 147^{th} cycle could be the application of 3 μA to electrode 5, the 148^{th} cycle could be the application of 3.1 μA to that electrode, the 148^{th} cycle could be the application of 2.7 μA to that electrode, the 149^{th} cycle could be the application of −0.4 μA to that electrode. And note that this does not mean that the difference must always be at least a certain amount, only that the device can establish that difference within the specified time period. Thus, providing that the cochlear implant can raise and/or lower the current applied to a given electrode by a sufficiently precise amount that is sufficiently large enough, within a sufficiently short period of time, the current source of the cochlear implant can be utilized to implement the voltammetry.

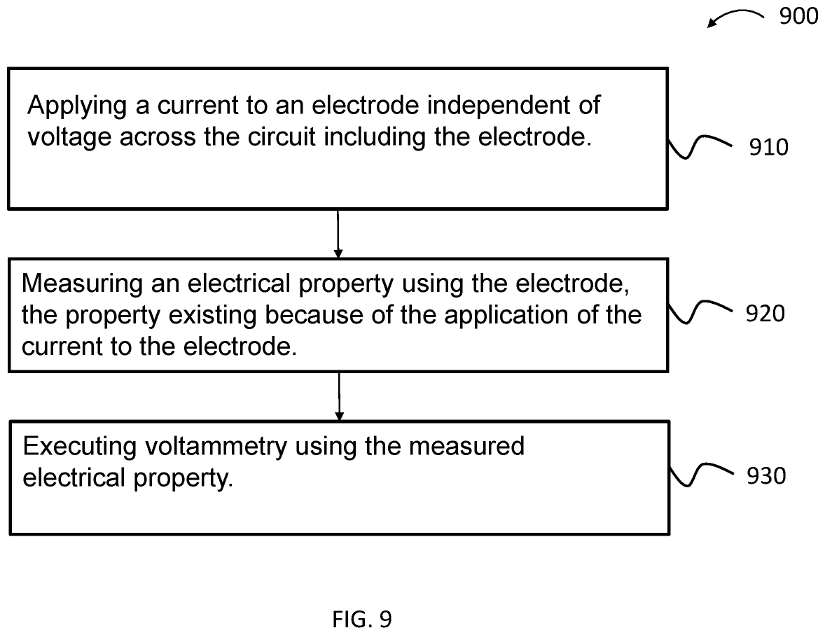

FIG. 9 presents an exemplary algorithm for an exemplary method, method 900. Method 900 includes method action 910, which includes the action of applying a current to an electrode independent of voltage across the circuit that includes the electrode. This can be executed utilizing a current source of a cochlear implant by way of example. Method 900 further includes method action 920, which includes the action of measuring an electrical property using the electrode, the property existing because of the application of the current to the electrode. In an exemplary embodiment, the electrical property has the potential between the electrode and another electrode, which can be the return electrode in some embodiments, such as an electrode located outside the cochlea, which can be an electrode located on a separate lead to separate from the lead that supports the electrode array of the cochlear implant, and/or can be a return electrode located on the housing of the receiver-stimulator. Concomitant with the teachings herein, the actions of applying current and measuring the electrical property can be executed with a device configured to generate the current and measure voltage as opposed to generate voltage and measure current. Note that method actions 910 and 920 can occur simultaneously. That is, the measurement of voltage can be executed while applying current to the electrode.

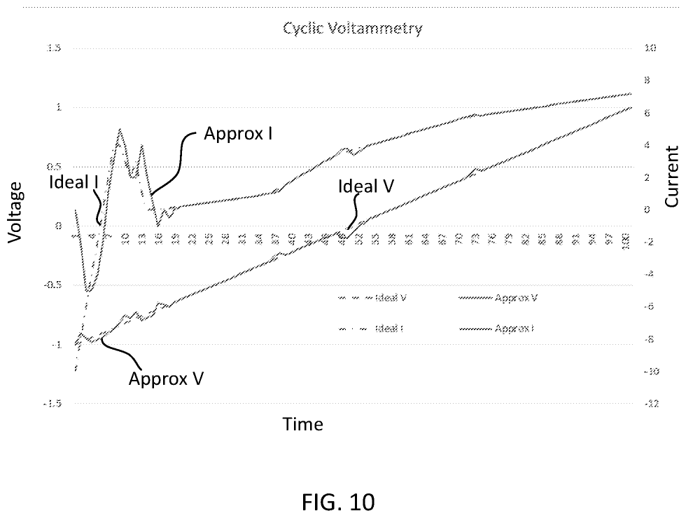

In an exemplary embodiment, the actions of applying a current and measuring the electrical property are repeatedly executed as part of a process that generates an approximation to a constantly ramping voltage at a reference electrode, wherein the reference electrode can be the electrode to which current is applied or another electrode. In this regard, some embodiments can provide a sufficiently accurate approximation of the voltage ramp this way because of the relatively slow speed of the voltage ramp and the long measurement times, coupled with the ability of cochlear implant current sources to generate rapidly varying current values and the ability of cochlear implant voltage measurement circuit to make high speed voltage measurements. FIG. 10 shows an example of Voltage (V) and Current (I) waveforms that can be generated by a control system according to an exemplary embodiment, used in a medical device such as a cochlear implant, or any other device that has a current source that can be controlled to implement the teachings herein, along with the ideal voltage ramp and ideal current waveforms. The timescale presented in FIG. 10 is a dimensionless timescale, but in some embodiments, the scale can be 0.5 1, 1.5, 2, 2.5, 3, 3.5, 4, 4.5 or 5 seconds or more or less or any value or range of values therebetween in 0.1 second increments. Again, some embodiments can be practiced with whatever time frames can provide utilitarian value. In an embodiment, the timescale represented can correspond to any one or more of the timeframes detailed herein (e.g., a few seconds, less than 4 seconds, etc.). The units of current are microamps. The units of voltage are volts.

Concomitant with FIG. 10 and FIG. 6, the action of applying a current in method action 910 is part of a process where a current applied to the electrode is varied upward and downward to achieve a desired voltage ramp. In some embodiments, the action of applying a current is part of a process where a constant current level is applied to the electrode in varying temporal lengths to achieve a desired voltage ramp. That is, with respect to these two variations, there are at least two ways to control the current source to achieve the desired voltage ramp which desired voltage ramp can approximate a real voltage ramp that would exist if a voltage source is being utilized for voltammetry in general, and cyclic voltammetry specifically.

With respect to the embodiment that utilizes the application of a current applied to the electrode in a varying manner upward and downward to achieve a desired voltage ramp, by way of example, the control system of the cochlear implant for example, can be configured to output current and measure voltage repeatedly, where the output of current is controlled based on the measured voltage. (The measurements can be taken in the same cycles as the current is outputted (e.g., if current is controlled in 1/1000 of a second cycles, voltage can be measured in 1/1000 second cycles as well) or can be less than the current cycle. That said, voltage measurements could be taken at a greater cycle rate than current change. Any implementation that will enable the teachings herein can be implemented providing the art enables such. As noted above, some embodiments can be configured to control current values at the sub-millisecond level. An exemplary embodiment exists where current can be controlled in sub-millisecond increments, but it is noted that embodiments can be implemented where that control is implemented at finer values or coarser values by way of example. The following is but an exemplary embodiment to explain the underlying concept, where it is to be understood that variations of this can and will exist providing that such can enable the teachings detailed herein.

In some embodiments, current is applied while also measuring voltage (current application and voltage measurement at the same time). That is, there is a shoot and look, shoot and look, shoot and look regime. In an embodiment, a current level can be applied at time interval 101, of X microamps. A voltage value of Y Volts is also recorded at time interval 101. Based on that voltage value, a current level is controlled to be applied at time interval 102 and voltage is measured, and then at interval 103, current is applied based on the measurement during interval 102, and voltage is measured. Based on the voltage value that is recorded at time interval 102, the current level that is controlled to be applied in interval 103 would be controlled to be an increase above X microamps or decrease below X microamps or the same for that matter as that which was the case during time interval 101. Conversely, in an embodiment, there is a look-shoot-look-shoot or shoot-look-shoot-look (or look shoot, shoot, look, shoot for example) implementation where current is not applied when measuring voltage. In an embodiment, a current level can be applied at time interval 101, of X mA. A voltage value of Y Volts is recorded at time interval 102. Note that the current could still be applied as in time interval 101. Based on that voltage value of interval 102, a current level is controlled to be applied at time interval 103, and then at interval 104, the voltage value is read. Based on the voltage value that is recorded at time interval 102, the current level that is controlled to be applied would be controlled to be an increase above X mA or decrease below X mA or the same for that matter as that which was the case during time interval 101. Also, the amount of that increase or decrease would also be controlled based on the voltage value read at time interval 102 (e.g., an increase of 5% of X mA, or a decrease of 3% of X mA, or even a decrease of 100% if needed if there is a voltage overshoot for example). This would also be the case with respect to the voltage value read at time interval 104. If for example, the process is at the point where the voltage is supposed to ramp up in a linear manner, if the voltage reading at time interval 104 indicates an overshoot over or an under shoot under the ideal voltage, the current will be adjusted accordingly in the next time interval (e.g., time interval 105) or the next possible time interval (it could be that an analysis of the voltage reading might take one or more time intervals—thus, the measurement of the time interval 102 could be what time the current interval in time interval 104 is based upon (at least in part—as will be described below, a PID can be used to control the current) to “keep” the voltage on track for the ideal voltage (or, more accurately, to steer the voltage back towards the ideal voltage), which ideal voltage in this exemplary embodiment should be a linear ramp up as shown for example in FIG. 10. The iterative nature of this can be seen in FIG. 10 where the approximate voltage curve deviates from the ideal voltage curve. Moreover, FIG. 10 shows the scenario that would result where a voltage source was being utilized to control the voltage and current was being read at the reference (the ideal curves), which is the opposite of that implemented according to the teachings detailed herein with respect to embodiments utilizing a current source.

To be clear, embodiments include keeping the current source energized (albeit the amount of current outputted being adjusted) continuously during the ramp up and/or ramp down period and varying the current level in response to the measured voltage(s). Embodiments include measuring voltage as quickly as possible or as quickly as utilitarian to implement the teachings detailed herein. The graph of FIG. 10 represents such an embodiment, where the current is continuously varied. In an embodiment, the voltage measurement cycles can be faster than the current application cycle and or vice versa, or can be the same. In an embodiment, current is varied as quickly as possible based one the analysis of the voltage readings. In this regard, in at least some exemplary embodiments, it is not enough just to read the voltages, the voltages must also be analyzed. Thus, there could be a lag between the measurement of voltage and the adjustment of current based on the voltage measured.

Embodiments thus include unbroken application of the power supply/current supply.

It is also briefly noted that embodiments include utilizing DC current to implement some or all of the teachings herein (note that simply because a current goes negative does not mean that it is alternating current). In this regard, the exemplary scenario depicted in FIG. 10 represents the application of the DC current from a given electrode. This is opposed to a biphasic stimulation or the application of a balanced current. That is, in an exemplary embodiment, balance of charge in tissue of the recipient is not taken into account with respect to the implementation of the voltametric techniques. This as compared to the application of alternating current to tissue, where there is no charge build up because the positive current is balanced by negative current. Here, there is no balancing. The charge (current multiplied by time) at the end of the ramp up and/or ramp down could be meaningfully different from zero or otherwise meaningfully different from that which would result from the application of alternating current. In an exemplary embodiment, the cochlear implant is configured to stimulate tissue utilizing the electrode utilizing alternating current such as to evoke a hearing percept. In this exemplary embodiment, the cochlear implant is also configured to switch to a direct current stimulation regime or otherwise direct current output mode to apply the voltammetry techniques detailed herein. Accordingly, embodiments include the application of firmware and/or software to existing cochlear implants to enable the cochlear implants to output current in a DC mode as well as an AC mode (the AC mode being utilized to evoke a hearing percept).

And while some embodiments include continuously energizing the current source and thus maintaining energizement of the electrode over the ramp up and/or ramp down period, other embodiments include continuously energizing the current source over a portion of the ramp up and/or ramp down period, and then implementing a different energizement regime (e.g., a shoot-look-shoot regime) from another portion of the ramp up and/or ramp down. Corollary to this is that in some embodiments, the constant current pulsatile arrangement can be utilized in combination with the continuously applied current. For example, during a first portion of the ramp up, the continuous application of current regime can be applied while in the second part of the ramp up, the pulsatile current regime can be applied, and so on. Embodiments include energizing/maintaining energizement of the current source and/or electrode with current for at least 50, 55, 60, 65, 70, 75, 80, 85, 90, 95 or 100% of the ramp up and/or ramp down time period. Voltage measurements are taken as desired or as needed or throughout the entire ramp up and/or ramp down periods.

Embodiments include devices, systems and/or methods that enable the determination of the “new” current source value based on the existing value, the desired voltage ramp voltage, and the measured voltage. In an exemplary embodiment, the device uses an algorithm that is based at least in part on the concept that the new current=old current+a constant×(desired voltage−measured voltage). Embodiments also include the utilization of proportional-integral-derivative (PID) controller techniques (embodiments can include a PID control techniques implemented using firmware and/or software in existing cochlear implants (but note embodiments include adjusting/adding hardware to an existing cochlear implant)). Embodiments can include the ability to store the current and/or voltage values at discrete temporal points in time in a memory to enable sophisticated control techniques.

Continuing with reference to FIG. 10, by way of example only, where the goal is to approximate the ideal voltage curves for voltammetry, the input can be the measured voltage where the error is the difference between the measured voltage and the desired voltage (the desired voltage being the ideal voltage, for example). The output can be the adjustment to the existing current level output or simply the new current level output (the system can recalculate the current each time and/or calculate the adjustment to the current current). Again, some embodiments utilize as many voltage measurements as possible (thus make measurements as fast as possible) and/or make as many adjustments as possible (make adjustments as fast as possible) to implement the voltammetry techniques herein. For example, as seen in FIG. 10, the current drops from roughly zero to −5 to drive the voltage (measured/approximate voltage) back downward towards the ideal voltage), and then as a result of undershooting the ideal voltage, the current is driven upwards towards 6, and then driven downward, and then upward a bit and then downward and so on. All the while the current is constantly applied and the voltages are being measured and the applied current is adjusted essentially continuously. Accordingly, the current source is continuously energized and the current level is varied in response to the measurements of the voltage (the variation being based on the given control algorithm used, PID or otherwise).

Other control techniques can be used. Any control algorithm that can enable the teachings detailed herein can be utilized in at least some exemplary embodiments providing that the art enable such.

It is noted that while some of the embodiments described above are described in terms of a energized-read-energized-read-energized-read and so on implementation, where the length of times for reading or equal to the length of times of energizement, in other embodiments, the length of energized and can be longer than the length of reading. By way of example only and not by way of limitation, 9 time periods can be dedicated to energizement, and the 10^{th} to reading. During those 9 time periods, the current can be constant or increased (or decreased) as would be estimated by the control system or other algorithm to achieve the desired voltage. In an embodiment, four time periods can be dedicated to energizement, and the fifth reading, or any other permutation that can have utilitarian value that can enable the teachings detailed herein. Indeed, the control system can be configured to determine the time periods for energizement and reading on an ongoing basis during the process. For example, if the voltage readings fall within a sufficiently narrow range above and/or below the ideal voltage readings, fewer voltage readings overtime might be implemented (in some embodiments) relative to that if one or more voltage readings were falling outside the sufficiently narrow range. That is, because the voltage readings are utilized to implement “course corrections” vis-à-vis controlling the current level applied by the current to the electrodes, the more “off course” the voltage readings from the ideal voltage, the more frequent the voltage readings can be in at least some exemplary embodiments, and vice versa.

The above said, in some embodiments, the system will make voltage measurements and current corrections as fast as possible or otherwise as fast as can achieve more than marginal utilitarian improvement, or any utilitarian improvement, to achieve the closest possible to a ramping voltage source.

Note further that the control system can be configured to account for the fact that a time interval has been applied for voltage reading instead of current application. In this regard, and an exemplary embodiment, the control system can be configured to adjust the next current level to be applied after the voltage reading to account for the fact that the voltage likely has dropped (or risen) during the time interval where the electrode was utilized for voltage reading. The amount of the change can be estimated based on prior knowledge of how the system operates in that given human based on general statistical values for the class of human or based on simple approximation of what will occur when current is no longer applied during that time interval, whether this approximation is based on the preceding ramp up or ramp down or some other factors. All of this said, such concepts can be adapted where current is applied while measuring voltage.

All the above said, it is noted that for the voltammetry teachings herein, a certain number of voltage readings and a certain number of current readings will be needed to obtain efficacious results. Because the device will be able to constantly read current in some embodiments, or otherwise, will know the current output at any given time interval because it is controlling the current output during those time intervals, the open variable will be the number of voltage readings that will be useful to achieve a sufficient set of data to implement voltammetry. Put another way, in some embodiments, there will be a baseline number of voltage readings that will be required to be taken based on the level of accuracy desired for a given voltammetry technique. Thus, even if relatively few voltage readings are needed to keep the approximate voltage within the desired range above and/or below the ideal voltage, more voltage readings than strictly necessary to keep the voltage curve within tolerable tolerances may still be taken to obtain the desired accuracy of the resulting data set.

The following exists by way of example only and not by way of limitation, where a hypothetical scenario of current adjustment over time so as to provide an example of the concept of controlling the current to vary the voltage to implement voltammetry according to the teachings detailed herein: I, 1.01I, 1.03I, 1.03I, 1.01I, 1.02I, 1.07I, 0.99I, 0.99I, 0.9I, 0.7I, 0.3I, 0.2I, −0.15I, −0.03I, −0.01I, 0.22I . . . . In this exemplary example, the percent change of I is based on the immediately preceding I. Thus, for example, if I equals 1 at the beginning (not that it is 1 amp or microamp—this is a base number for purposes of explanation only), the next I would be 1.01, and then the next I would be 1.0403, and the next would be 1.0715, and so on. And note while this embodiment utilizes percentages as the adjustment factor, in other embodiments, it can be a pure predetermined set amount current value that can be adjusted. That is, by way of example, the current value can be increased by a predefined amount each time, such as, for example 0.001 milliamp. Because in at least some exemplary embodiments, the period of time for each cycle is so short and thus the change in current can take place relatively fast, in some embodiments, the predefined amount can be utilized. And when there is a determination that there is a voltage overshoot for example, a lower current can be applied than that previously applied (or no current can be applied in embodiments where the current is pulsatile) until there is a determination that there is an undershoot, and then the current can be applied to increase the read voltage to the ideal level, at which point a reading indicating such (or an estimate that such is the case—in an embodiment, the control system can be configured to analyze a level of the current that is applied and otherwise estimate what the voltage reading will be instead of taking a reading at that point in time, or in addition to taking a reading at that time, where the estimates for the voltage level can be utilized until a reading takes place, and then the reading can be utilized as a basis for future estimates) would warrant applying a lower current (including a negative current) or not applying current (for a pulsatile arrangement) for one or more cycles, and so on.

And note that this example did not provide an interval for voltage readings. In at least some exemplary embodiments, there will be intervals between the applied current values in the example above where no current is applied but the electrode is utilized to read a voltage because it is necessary to read the voltage to obtain values for “course correction” and also simply to obtain voltage reading values to implement the voltammetry teachings herein because in the end, voltage values are needed as part of the data set. In an exemplary embodiment, the system can be a “smart system” where, for example, if the system determines that little to no current should be applied, the system can apply no current and instead use that time interval as a reading. Thus, the system could “bite the bullet” during time intervals where little current is needed to maintain the approximate curve close to the ideal curve. In an embodiment, this could throw off the curve abet because the system would know that some current is still needed, but in the greater scheme of things, not applying current during one time interval has more utilitarian value then not doing so in another time period where current might be needed to keep the approximate curve on track with the ideal curve.

It is further noted that in an exemplary embodiment, negative current could be applied as needed during one or more time intervals to adjust the approximate voltage curve to more closely align with the ideal voltage curve. Thus, while the exemplary conceptual embodiment described above only shows at a minimum the application of zero current, it is to be noted that in some embodiments, a negative current can be applied as needed. Any regime of current control of current from a current source that can enable the teachings detailed herein can be utilized in some embodiments providing that the art enable such. Any control regime that can establish an approximate voltage curve that sufficiently corresponds to an ideal voltage curve for voltammetry that can enable the teachings detailed herein can be utilized in at least some exemplary embodiments again providing that the art enable such.

The above examples focused on the utilization of changing a current level during given energizement periods to obtain an approximate voltage that sufficiently parallel the ideal voltage to implement voltammetry. In other embodiments, and it is noted that these embodiments are not mutually exclusive unless otherwise noted or unless the art does not enable such, it is the length of time that a given current is applied that is varied to obtain an approximate voltage that is sufficiently parallel to the ideal voltage to implement voltammetry. By way of example, with reference to the concept of energizement cycles, a relatively high current level can be applied to the electrode for a certain number of cycles to “bring up” the voltage at the electrode, and then there can be cycles without current application to the electrode until a determination is made that additional current should be applied to elevate the voltage at the electrode. And note that the reverse is also true: if the goal is to reduce the voltage level at an electrode by a certain amount (and corollary to this is that in at least some embodiments, by no more than a certain amount—note that embodiments disclosed herein work because the ability to control current into control voltage in precise amounts to implement voltammetry utilizing a current source instead of a voltage source), current will be withheld or can be withheld for certain number of cycles until that voltage reduction is met, after which current can be applied for a certain number of cycles, and so on.

Relative to that which would otherwise be the case if the current was maintained for a longer period of time. For example, if a current value of Z were applied for 100 ms, the voltage would be unacceptably high (unacceptable vis-à-vis achieving the approximate voltage curve that is sufficiently accurate relative to the ideal curve), but if that current value of Z is applied for only 20 ms or 15 ms (or 17 ms, or however long is needed to achieve the desired voltage) for example, the resulting voltage would be acceptable and otherwise would correspond to the approximate voltage that sufficiently accurate relative to the ideal voltage curve.

Accordingly, by way of example, an energizement regime could be ZI, ZI, ZI, 0, ZI, ZI, ZI, ZI, 0, 0, ZI, 0, 0, 0, ZI, 0, ZI, 0, ZI, ZI, ZI, ZI, ZI, ZI, ZI, 0, ZI, 0, ZI, ZI, ZI, ZI, ZI, 0, 0, 0, 0, ZI, ZI, 0, 0, 0, ZI, 0, ZI, 0, ZI, ZI, ZI, ZI, 0, ZI, 0, ZI, ZI . . . and so on. And note that the zero periods could be voltage read periods. Note that the energizement periods would be contiguous and continuous until zero voltage. Thus, for example, the first part of the above before the first zero would be three (3) milliseconds of ZI without interruption until the current is cut off at the 4^{th} millisecond (assuming for this example only that the periods are 1 ms long—as will be described in greater detail below, the periods can be of different lengths). And if the ZI were maintained for more periods, the resulting voltage would be sufficiently accurate with the ideal voltage.

By rough analogy, this regime can be like a bird flapping its wings, where the bird only flaps to increase altitude or to maintain altitude, and the force of the flapping is the same, it is only the number of times that the wings flap that are different. This as compared to a bird that has a variable force with respect to each flap, which would more accurately the analogized to the regime detailed above where current levels are varied.

Accordingly, embodiments can include pulsing a fixed current various amounts over time and/or controlling an amount of current given during time.

In view of the above, it can be seen that in some embodiments, with respect to the action of applying a current invariant temporal lengths, the varying temporal lengths can be in the 10^{th} of a second range, 100^{th} of a second range, 1000^{th} of a second range, 10000^{th} or 100000^{th} 1000000^{th} of a second range, or less. That is, the applied current can be varied in a controlled manner over such increments (by the amounts that the device can adjust per those times, such as by the times detailed above). Put another way, the circuits of the device that is utilized to apply the current can be such that the current can be controlled within the aforementioned ranges to achieve the desired voltages. Conversely with respect to a pulsatile arrangement, the circuits of the device can be turned on and then shut off to effectively zero within these aforementioned ranges. That is, the applied current can be applied in such increments and controlled to be not applied in such increments. Put another way, the switching circuits of the device that is utilized to apply the current can be such that the current can be applied at the maximum amount and then shut off to effectively zero within these aforementioned ranges. It is this ability to quickly apply and remove current and/or, in the regime detailed above with respect to varying the current, the ability to quickly apply a certain amount of current and then reduce the current by a certain amount and/or increase the current by a certain amount in a controllable manner within the temporal periods just detailed, that enables the utilization of the current source to implement voltammetry instead of the traditional utilization of a voltage source.

In an embodiment, the current source of the device is configured so that the current applied to the electrode can be varied upward and/or downward by amounts of at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 350, 400, 450, 500, 600, 700, 800, 900, 1000, 1250, 1500, 1750, 2000, 2500, 3000, 4000, 5000% or more or any value or range of value therebetween in 0.01% increments within a tenth of a second and/or within 100^{th} of a second and/or within 100,000^{th} or 1,000,000^{th} of a second (or some increments in between—any increment within 1,000,000^{th} of a second or any range therebetween) to achieve a desired voltage ramp. (Note that the variation can be less than the aforementioned amounts—indeed, embodiments can be configured to vary the current by amounts between 0.0001% and 0.1% or any value or range of values therebetween in 0.0001% increments. And note that this does not mean that the increments are tenths of a second or 100th of a second. What this means is that the current must be varied by at least 10% within that time (and the value of the current is the average of the current over that time) the increment becomes the denominator for time). How that is done can be any of the teachings detailed herein or any variations thereof or other techniques providing that such enables the method.

Accordingly, in an exemplary embodiment, for a given time period less than and/or equal to A, greater than and/or equal to B amount of current can be controllably applied within C % of the desired amount. A can be less than and/or equal to and/or greater than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 15, 30, 35, 40, 45, 50, 60, 70, 80, 90, 100, 120, 130, 140, 150, 160, 170, 180, 190, 200, 225, 250, 275, 300, 350, 400, 450, 500 ms or more or any value or range of values therebetween in 0.001 ms increments. B can be less than and/or equal to and/or greater than 0.1, 0.2, 0.3, 0.4, 0.5, 0.6, 0.7, 0.8, 0.9, 1, 2, 3, 4, 5, 6, 7, 8, 9, 10, 15, 20, 15, 30, 35, 40, 45, 50, 60, 70, 80, 90, 100, 120, 130, 140, 150, 160, 170, 180, 190, 200, 225, 250, 275, 300, 350, 400, 450, 500, 600, 700, 800, 900, 1000, 1100, 1200, 1300, 1400, 1500, 1600, 1700, 1800, 1900, 2000, 2250, 2500, 3000, 3500, 4000, 4500 or 5000 microamps or more or any value or range of values therebetween in 0.01 microamp increments. The aforementioned time periods are not the power cycles, although the time periods above can mesh with the power cycles and can be power cycles. But for clarity, the power cycles (the time in which current can be controlled) can be less than, greater than and/or equal to 30, 40, 50, 60, 70, 80, 90 100, 150, 200, 250, 300, 350, 400, 500, 600, 700, 800, 900, 1000, 1100, 1200, 1300, 1400, 1500, 1600, 1700, 1800, 1900, 2000, 2250, 2500, 3000, 3500, 4000, 4500, 5000, 5500, 6000, 6500, 7000, 8000 or more Hz or any value or range of values therebetween in 0.1 Hz increments, and it is within these power cycles that the current is controlled.

And as noted above, for a given time period A, B amount of current can be controllably applied within C % of the desired amount, where C can be 80, 85, 90, 91, 92, 93, 94, 95, 96, 97, 98, 99 or 100% or any value or range of value therebetween in 0.1% increments, except where the current is to be zero, in which case it is no more than 10, 9, 8, 7, 6, 5, 4, 3.5, 3, 2.5, 2, 1.5, 1, 0.9, 0.8, 0.7, 0.6, 0.5, 0.4, 0.3, 0.2 or 0.1% or less or any value or range of values therebetween in 0.01% increments from that which was the case the last cycle or time period where current was not zero. Put another way, the aforementioned percentages provide a way to quantify the ability to control current. For example, not all arrangements that ultimately apply and prevent the flow of current may not be sufficiently precise or accurate to implement the teachings detailed herein. Simply because current is applied to an electrode does not mean that that current is applied within the parameters needed to implement the teachings detailed herein, and corollary to that, is that simply because current is prevented from reaching the electrode, or otherwise the application of current is stopped, does not mean that it is stopped swift enough or by amounts sufficient enough to implement the teachings detailed herein.

By way of example, in the case of pulsing current at fixed levels, if during a given time period current is to be applied at value I, where, for example, the given time period lasts for example 3 cycles, if the first cycle only reaches 50% I for example, and in the second cycle only reaches 75% I, the result may not be sufficient to achieve the desired voltage. Conversely, if the first cycle reaches 95% I for example and the second cycle reaches 99%I, the result is likely to be sufficient to achieve the desired voltage. Corollary to this is if the fourth cycle requires zero current, and instead there is 23%I (the value of by the preceding cycle) again, the regime may not work to achieve the desired voltage.

Again, the teachings herein work because of the ability to precisely control current with in precise and short periods of time.

And with regard to the approximate volage corresponding sufficiently to the ideal voltage, in an embodiment, the mean, median and/or modal variation from the ideal voltage (ideal being the denominator) is no more than 10, 9, 8, 7, 6, 5, 4, 3.5, 3, 2.5, 2, 1.5, 1, 0.9, 0.8, 0.7, 0.6, 0.5, 0.4, 0.3, 0.2 or 0.1% or less or any value or range of values therebetween in 0.01% increments. In an embodiment, the mean, median and/or mode of the voltage is taken over the entire ramp up and/or ramp down, ½, ⅓, ¼, ⅕, ⅙, 1/7, ⅛, 1/9, 1/10, 1/11, 1/12, 1/13, 1/14, 1/15, 1/16, 1/17, 1/18, 1/19, 1/20, 1/25, ⅓, 1/35, 1/40, 1/45, 1/50, 1/60, 1/70, 1/80, 1/90 or 1/100^{th} or finer or any value or range of values in 1/1000 increments of the ramp up and/or ramp down, and each portion need not have the same percentage. For example, in the first quarter of the ramp up, the mean variation could be no more than 5.54%, and in the second quarter of the ramp up, the mean variation could be no more than 1.3%, and so on (where the denominator is the ideal voltage). There can be utilitarian value with respect to dividing up the ramp up as just described, because, for example, the beginning of the ramp up could be the most loosely correlated portion owing to the fact that the control system is iteratively figuring out what current to apply to achieve the desired voltage, and once that becomes more stabilized otherwise more predictable, and thus better controlled, the variation between the ideal voltage and the approximate voltage would be likely to be reduced with temporal progression.

Note that the above values can be also for the ideal current vs. the approximate current (ideal is the denominator). And note that the values need not be the same as that for the voltage. Note further that any value given herein can be applicable for one set of circumstance and another value for another. Nothing need be the same unless specifically noted providing that the art enables such.

In view of the above, there is an exemplary embodiment that includes a medical device where the implantable portion of the medical device includes at least one electrode, wherein the implantable portion includes a current source (as distinguished from a voltage source), and has as its principle of operation controlling current from the current source to stimulate tissue of a human to evoke a reaction associated with the tissue (where controlling can be controlling the current source and/or controlling what flows from the current source to the electrode). In this embodiment, the medical device (which means that it need not be the implantable portion—as noted above, the implantable portion can be controlled, and note that it need not be the external component of the prosthesis (e.g., the BTE device)—the medical device can include a dedicated component to do the following, such as a dedicated surgical instrument that controls the implant, such as that which might be used during surgery or after surgery but before activation of the cochlear implant for hearing purposes, for example) is configured to control current from the current source (again, by controlling the current source or some other parameter that controls the current to the electrode) to establish a specific electrical phenomenon in reaction to the control of the current source and obtain data based on the electrical phenomenon, which electrical phenomenon and data is sufficient to execute voltammetry, such as, for example, cyclic voltammetry. In an exemplary embodiment, the results of such can correspond to any one or more of those detailed herein (e.g., the mean values for the ramp up and/or ramp downs noted above, for example).

In an embodiment, the medical device includes a control system configured to approximate an ideal CV system using hardware of a current source, and, in some embodiments, the medical device is a cochlear implant. In some embodiments, the medical device is configured to make rapid measurements of voltage at the electrode or another electrode and, based on the measurements, vary current magnitude and/or temporal length of application of current to approximate an ideal CV system.

Consistent with embodiments where a commercial of the shelf cochlear implant is utilized to implement at least some of the teachings detailed herein, in an exemplary embodiment, the medical device is of a design where a software change can completely eliminate the ability to establish the specific electrical phenomenon. Corollary to this is that a software change can implement or otherwise enable the ability to establish the specific electrical phenomenon.

Embodiments include arrangements where the implantable portion is configured to output data based on data indicative of temporally correlated values of current supplied to the electrode from the current source and temporally correlated voltage readings of at least D values and at least D readings per second (where the two need not be the same), wherein D can be a wide number, such as 5, 10, 20, 30, 50, 100, 500, 1000, 5000, 10000, 20000, 30000, 40000 50000, 60000, 70000, 80000, 90000, 100000, 125000, 150000, 175000, 200000, 25000, 300000, 35000, 400000 or 500000 or more or any value or range of values therebetween in 1 increments (e.g., 33, 66, 77 to 333, etc.).

In an embodiment, the implantable portion includes an electrode that is separate from electrodes used by the device in normal stimulative operation, which electrode that is separate is a reference electrode, and wherein the separate electrode has a relatively stable rest potential, and wherein the separate electrode is coupled to a high input impedance measurement amplifier. In this regard, it is noted that most embodiments described above have been directed towards the concept of utilizing an electrode of an electrode array, or otherwise a working electrode of the medical device and the standard return electrode. These two electrodes would operate in a source and sink arrangement in some embodiments. Conversely, in this embodiment with the separate electrode, there is an electrode that can be used as a dedicated electrode for the purposes of implementing voltammetry. Note that this is not just the electrode that might be located on the housing of the receiver-stimulator, which can be in addition to the so-called “hard ball” electrode that extends from a lead from the housing separate from the lead that extends from the housing to the electrode array, although embodiments could certainly implement such. This electrode under discussion in this embodiment is an electrode that is intended to be used only for the purposes of voltammetry. In an embodiment, this electrode could be an extra cochlear electrode, and would be a return for any one or more of the electrodes of the electrode array when those electrodes are utilized to execute voltammetry.

As noted above, a high impedance amplifier could be used. In an embodiment, it is utilitarian to essentially prevent current from traveling through the electrode. In an embodiment, the electrode can be made as large as possible to minimize tissue influence. Consistent with the teachings herein, the electrode could be made out of platinum or titanium. In an exemplary embodiment, this electrode is never used for tissue stimulation. That said, in an exemplary embodiment, the implant can be configured so that if there is ever a need in the future to utilize this electrode for tissue stimulation, the aforementioned high impedance amplifier can be bypassed or otherwise shut down, and the electrode could be utilized. In essence, this electrode could be provided for the purpose of implementing voltammetry, but could also be utilized as a spare extra cochlear electrode if such was ever needed. This could prevent or otherwise reduce the utilitarian value of this electrode for voltammetry, but that could be a small price to pay when compared to the scenario of not being able to use the implantable portion at all in the event that this extra cochlear electrode is needed for tissue stimulation.

Thus, by way of example, this could create a three-electrode system where, for example, the electrode of the electrode array is a working electrode, the standard return electrode is the reference electrode, and the additional electrode is the auxiliary electrode. That said, the additional electrode can be used in a two-electrode system.

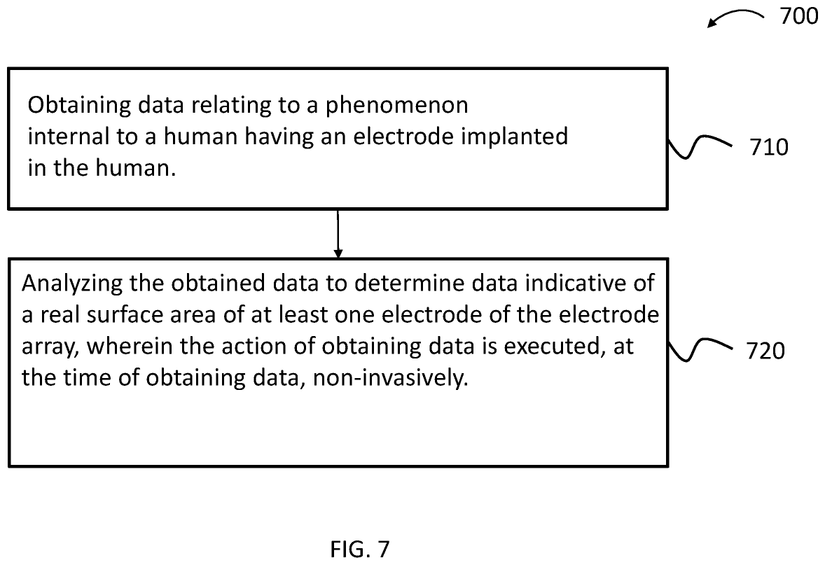

FIG. 7 presents an exemplary algorithm for an exemplary method, method 700, according to an exemplary embodiment. Method 700 includes method action 710, which includes the action of obtaining data relating to a phenomenon internal to a human having an electrode (e.g., as part of an electrode array) implanted in a human. In an exemplary embodiment, the electrode is part of an electrode array is a cochlear implant electrode array implanted in a cochlea. In an exemplary embodiment, the obtained data is voltammetry data, such as, for example, cyclic voltammetry data.

In some embodiments, the phenomenon is based on current and/or voltage measurements associated with at least one electrode (e.g., an electrode of the array), wherein the electrode is implanted in a human, such as an electrode of an array that is a cochlear implant electrode array implanted in a cochlea. But again, embodiments can utilize the techniques herein with respect to a retinal implant or a pacemaker electrode or a muscle stimulator electrode or a brain stimulating electrode, or a sensor that is utilized to sense brain activity such as a seizure sensor for a person afflicted with epilepsy. Embodiments can be such that the obtained data is current vs. potential data for a varying potential of the at least one electrode of the electrode array, concomitant with the voltammetry teaching herein.

Method 700 further includes method action 720, which includes analyzing the obtained data to determine data indicative of a real surface area of the electrode. In this embodiment, the action of obtaining data is executed, at the time of obtaining data, non-invasively. In this regard, such as where the obtained data is obtained utilizing the implanted portion of the cochlear implant, even though an invasive procedure was required to implant the implantable portion, because the method is qualified at the time of the action of obtaining data, if this occurs after implantation/after the implantation procedure is completely completed, the data is obtained non-invasively. In an exemplary embodiment, the action of obtaining data is executed, again at the time of obtaining data, minimally invasively. In this regard, as opposed to the action of implanting the implantable portion of the cochlear implant of FIG. 1A, which would be an invasive procedure, a needle to access, for example, a blood sample, such as accessed from a vein in one's arm, would be minimally invasive.

Briefly, with respect to data indicative of real surface area, this can correspond to a percentage or some value relative to the virgin electrode/electrode at implantation.

In an exemplary embodiment, any one or more of the actions detailed herein associated with electrode maintenance/preservation, etc., can be executed upon a triggering event associated with the wear status and/or wear rate of the electrodes. In an exemplary embodiment, any one or more of the actions detailed herein are triggered upon a determination that the wear status of the electrode is greater than, less than and/or equal to 99, 98, 97, 96, 95, 90, 85, 80, 75, 70, 60, 50, 40, 30, 20, 150, 14, 13, 12, 11, 10, 9, 8, 7, 6, or 5 percent, or any value or range of values therebetween in 1% increments of the total value of the virgin electrode (e.g., mass, volume, etc.). In an exemplary embodiment, any one or more of the actions detailed herein are triggered upon a determination that the wear rate of the electrode is greater than and/or equal to 30, 20, 15, 10, 9, 8, 7, 6, 5, 4, 3, 2, or 1% or any value or range of values therebetween in 1% increments per year and/or per decade.

In an exemplary embodiment, method action 720 can be executed in an automated and/or a manual fashion. As noted above, the neural networks and/or a trained expert system can be utilized to automatically analyze the obtained data. Moreover, in some embodiments, the action of analyzing is executed automatically by a prosthesis of which the electrode array is a part (e.g., by the implantable portion of the cochlear implant and/or by the external component of the cochlear implant, etc.). Accordingly, there are computing devices and/or medical devices along these lines that are configured to execute method action 720.

In some embodiments, the action of analyzing includes evaluating a portion of the current vs. potential (where such is obtained as noted above) that is proportional to the real surface area of the at least one electrode. Again, embodiments can utilize the teachings detailed herein to evaluate the wear of the electrode in question, or otherwise the dissolution states thereof, so as to determine an efficacy of that electrode these of the utilization of that electrode for the intended functionality thereof, such as evoking a hearing percept in the case of a cochlear implant, or monitoring or otherwise pacing a heart with respect to a pacemaker, etc. The idea is that by determining the real surface area of the electrode, the state of the electrode can be ascertained. Remedial action can then be taken place if such is needed, which remedial actions are detail below by way of example. Embodiments can include a method that includes the action of prescribing a substance to be ingested by the human to slow a future rate of wear of the electrode and/or prescribing a substance to be injected by the human that has an effect on the future rate of wear of the electrode. In an embodiment, the stimulation parameters can be changed to reduce a rate of dissolution (or to even prevent any further meaningful dissolution).

As with all of the actions of analyzing detailed herein unless otherwise noted, the action of analyzing can be executed automatically and/or manually, by the prosthesis or by a device in communication with the prosthesis, or by a device that is remote—note that the action of obtaining data otherwise that the obtained data includes data that was developed by third-party. In this regard, method 700 can be executed by a party that did not operate the prosthesis or otherwise engage in controlling the prosthesis to implement, CD for example. Instead, the underlying data could be developed by a third party, such as the recipient of the cochlear implant, or autonomously by the prosthesis, and this data can be transmitted or otherwise obtained by a specialist, the attention of the data by the specialists corresponding to method action 710. And it is noted that all of the actions of obtaining detailed herein can be executed automatically and/or manually, again by the prosthesis and/or by device in communication with the prosthesis which device could control the prosthesis to execute the CV actions for example to develop the underlying data. Still, embodiments are envisioned where the prosthesis controls itself to periodically execute CV, and then the data is accessed at some point in the future which access could be real time or could be delayed by number of days or months even.

Artificial intelligence can be utilized or otherwise a trained neural network or an expert system can be utilized to execute the analyses herein. The action of analyzing the obtained data can include determining the wear rate and/or wear status of the electrode(s). The action of analyzing the obtained data can also include determining a believed causation of the passive dissolution and/or active dissolution of the electrode. The action of analyzing the obtained data can include determining the presence of a given chemical or the presence of blood, etc., within the cochlea. The action of analyzing the obtained data can include determining that certain proteins or some other substances or is not present. In this regard, if there is an infection or the like, or other certain organic reactions occurring, this will produce different proteins in the perilymph. Different infections will produce different proteins. In any event, this will change the chemical makeup or otherwise the state of the perilymph. This will induce bumps or changes in position of the various inflection points on the CV curve, such as the CV curve of FIG. 5. By evaluating the bumps and/or comparing the locations to data for a body fluid of a human that is not experiencing an infection or some form of reaction, etc., a determination can be made about the state of the body fluid or otherwise that there is an infection or the like. This concept is also applicable to the various chemicals that may or may not be present in the perilymph for other body fluid owing to the drugs that a human is taking, etc. Accordingly, embodiments include evaluating the CV curve or otherwise the voltammetry data for telltale fingerprints along the curve that indicate a certain phenomenon is taking place or otherwise has occurred. Embodiments further include taking action based on those determinations that the phenomenon is taking place or otherwise has occurred, which actions can correspond to any of those detailed herein is applicable.

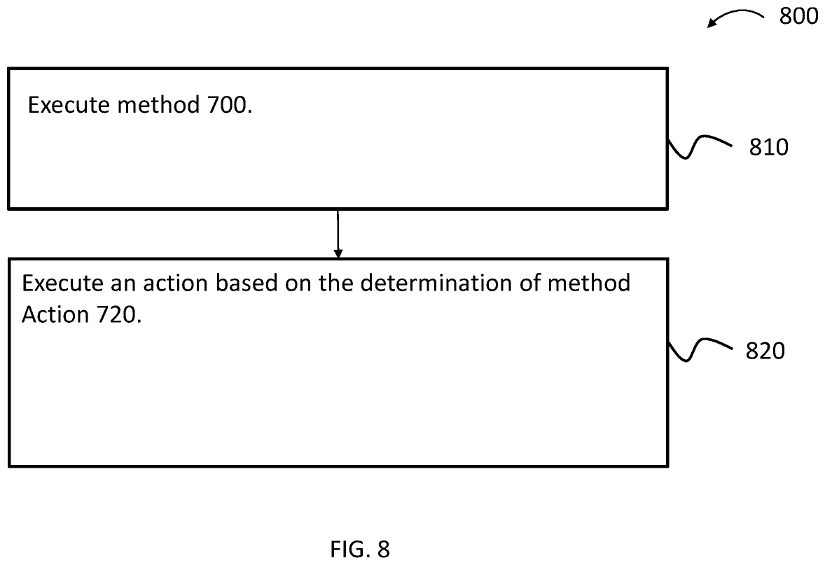

FIG. 8 shows an algorithm for an exemplary method, method 800. Method 800 includes method action 810, which includes the action of executing method 700 detailed above. Method 800 further includes method action 820, which includes taking an action based on the determination of method action 720. In an embodiment, the action of method action 820 can be adjusting a parameter of a cochlear implant based on the determination, wherein the electrode array is a cochlear implant electrode array of the cochlear implant. In an exemplary embodiment, method action 820 can correspond to identifying an adjustment of an operational parameter of the medical device prosthesis to change, such as to slow a future rate of passive dissolution and/or active dissolution. In an exemplary embodiment, this can entail reducing a current applied by the electrode and/or can include using a lower current combined with a longer pulse width to achieve a constant perceived level. Again, as promised, some additional ways to slow the future passive dissolution rate and/or active dissolution rate will be described.

Note also that the action of analyzing the obtained data can also include determining that there is no active dissolution and/or passive dissolution/the analysis can be to determine that there is no active dissolution and/or passive dissolution, and/or that any passive dissolution and/or active dissolution is de minimis, at least with respect to the ultimate timelines associated with need of the electrode (if the electrode wear rate that is determined would result in the electrode wearing out decades after the expected life expectancy of the user, such wear would be de minimis). In an exemplary embodiment, if indeed there is no passive dissolution and/or active dissolution or otherwise the rate is de minimis, method action 820 can result in action can be taken that increases a wear rate but also increase the efficacy of the prostheses. This too would be a result of implementing an identified adjustment of an operational parameter of the medical device prostheses to change a future rate of passive dissolution and/or active dissolution. By way of example only and not by way of limitation, an increase in current amplitude and/or an increase in focusing of a multipolar stimulation can be used in at least some exemplary embodiments. While this may result in an increased wear rate, this can also result in superior performance results, such as the ability to evoke a hearing percept that more accurately represents normal hearing (how a person with normal hearing hears).

Method action 820 can also include the action of prescribing a substance to be taken by the human or given to the human (e.g., ingested, injected, inhaled, etc.) to slow the future rate of passive dissolution and/or active dissolution and/or prescribing a substance to be taken by the human that has an effect on the future rate of passive dissolution and/or active dissolution. Such substances could change the chemistry of body fluids, such as a chemistry of the perilymph, that has an effect on the passive dissolution and/or active dissolution rate.

In some embodiments, the recipient can be directed or otherwise adjusted to change treatments for certain conditions. Counseling can be provided regarding the use of drugs found to have a deleterious effect on the rate of passive dissolution and/or active dissolution of an electrode. Conversely, a recipient can be instructed or otherwise encouraged to utilize certain therapeutic substances, such as drugs, over-the-counter or prescriptive, etc., that can have a positive effect on the biochemistry of the recipient so as to reduce, which includes halting, the dissolution of the electrodes.

Thus, an embodiment includes executing method action 820 by adjusting a functional component of a medical device of which the electrode is a part based on the determination.

In an exemplary embodiment, the action of adjusting a functional component of a medical device of which the electrode is a part based on determination is such that the action of adjusting is executed, as measured from a date of implantation of the electrode, within and/or equal to 5, 10, 15, 20, 25, 30, 35, 40, 45, 50, 55, 60, 65, 70, 75, 80, 85, or 90%, or any value or range of values therebetween in 1% increments of a reliability engineering based average (mean, median and/or mode) design life expectancy of the implantable portion of the medical device. Reliability engineering based design life expectancies are not specifically usage based. That is, one takes the design and determine the life expectancy based on normal usage. If abnormal usage occurs, the reliability might be recalculated, but that is usage based reliability. Thus, for example, for a model ABC prosthesis, the reliability engineering based average design life expectancy could be 75 years (can be the case for an implantable component of a hearing prostheses such as a cochlear implant). The reliability engineering based average design life expectancy could be 50 years for pacemaker (e.g., because people would require pacemakers later in life). This is all as contrasted to, for example, that which may be the case if the implantable component is utilized in an abnormal manner. By way of example only and not by way of limitation, if a user of the cochlear implant always has the volume to the maximum, the current amplitude of electrical signals applied to the electrodes will be higher on average than that which would otherwise be the case. This could cause the electrodes to wear faster than the average user of a cochlear implant. Reliability engineering data can exist for such usage, but that is not design usage, as the implant is not designed for use at a maximum volume for 50 years, even though it could be utilized at a maximum volume for a relatively long time, and there could be a possibility that the device would not wear out within that time period.

With respect to the above noted percentages of the life expectancy, if, for example, a cochlear implant has a life expectancy of 50 years, or more accurately, if the implantable portion of the cochlear implant, has a life expectancy of 50 years, or even more accurately, if the electrode array of the cochlear implant has a life expectancy of 50 years (the receiver stimulator of the cochlear implant could potentially be replaced without disturbing the electrode array or otherwise replacing the electrode array—moving the electrode array could cause problems with respect to the cochlea's accommodation of an existing array—a new receiver stimulator could be implanted and attached to the old electrode array in general, and the leads coming from the electrode array in particular, in at least some exemplary scenarios), and the adjustment is made within 10% of the reliability engineering based average design life expectancy of the implantable portion and/or the electrode array for that matter, the adjustment would be made within five years. The adjustment could be made within two years or three years.

In an exemplary embodiment, such as where the medical device is a cochlear implant, method action 820 can include instructing the user to use the medical device in a different manner. For example, the instruction for use of a cochlear implant in a different manner can be the reduction of a proportion of time and/or a number of listening environments in which focused stimulation is delivered by the cochlear implant. In an exemplary embodiment, the instruction is to simply reduce the amount of time that the implant is utilized per day and/or per week, etc. In an exemplary embodiment, the instruction is to maintain a volume as low as possible or otherwise adjusted volume to a level lower than that which has historically been utilized by the recipient. In an exemplary embodiment, the instruction is to utilize stimulation modes that adopt a monopolar and/or a bipolar stimulation regime whenever possible or otherwise more than that which has historically been the case. In an exemplary embodiment, the instruction is to utilize as a default the stimulation regimes of lower complexity and/or less focus, and use the stimulation regimes of higher complexity and/or greater focus only on special occasions or otherwise as needed.

In an embodiment, method action 820 includes instructing the human recipient of the medical device to use a medical device prosthesis that includes the electrode in a different manner, wherein the medical device prosthesis is a cochlear implant and the use of the cochlear implant in a different manner is reducing a proportion of time and/or a number of listening environments in which focused stimulation is delivered by the cochlear implant.

Of course, some exemplary methods include actually implementing the recommended changes. That is, the actions of method action 820 can include the affirmative actions resulting from the identification and/or prescribing and/or proscribing and/or instructing. For example, instead of identifying an adjustment and/or including identifying an adjustment, the adjustment is also made. It is also noted that in an exemplary embodiment, method action 820 can include taking no action. In many exemplary scenarios, the method actions herein will be executed where the analysis of the obtained data results in a finding that the electrodes are not dissolving and/or eroding or otherwise that the rate of passive dissolution and/or active dissolution is de minimis. As will be detailed below, the method actions herein of method 700 can be executed repeatedly over the life of the implant.

In an exemplary embodiment where method action 820 entails identifying an adjustment of an operational parameter of the medical device prostheses to slow a future rate of passive dissolution and/or active dissolution, where the medical device is a cochlear implant, the adjustment is a reduction in a degree of focusing for at least one channel of the cochlear implant which channel uses the electrode.

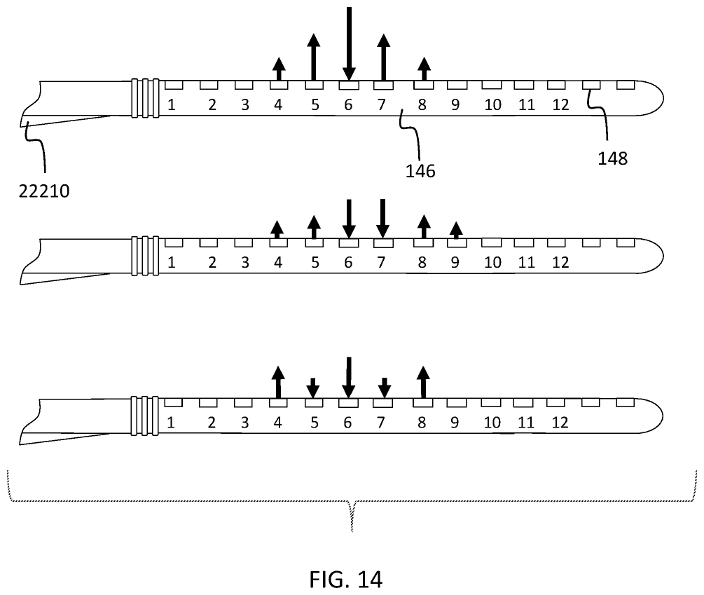

In an exemplary embodiment, say, where the results of the analysis of method action 820 results in a determination rate that the dissolution rate on an electrode is too high (e.g., the current rate will result in the electrode dissolving before the end of the recipient's life), the stimulation is spread out in a focused multipolar mode or other modes to more than one electrode. FIG. 14 presents a schematic symbolically representing how current could be redistributed in a focused multipolar mode. As can be seen, there is an electrode array with a plurality of electrodes, of which 12 have been enumerated for the purposes of this discussion. It is to be noted that this is simply exemplary and that the actions associated with one electrode can be applicable to the other electrodes. It is also to be understood that other embodiments can handle a given electrode differently. In any event, it is noted that the size and directions of the arrows indicate the magnitude and direction of current, respectively. The top electrode array depicts current flow in a normal or otherwise an optimized setting (optimized for that particular recipient vis-à-vis the hearing percept evoked in the person—as distinguished from, for example, battery life or electrode longevity for example). Upon executing method action 1820, it is determined that the electrode 6 is experiencing a wear rate which is higher than that which is deemed acceptable. The focusing of the multipolar currents is adjusted to that seen in the middle electrode array. This has the effect of reducing the wear rate on electrode 6.

The bottom electrode array also depicts optimized focused multipolar currents for the optimization of a hearing percept. Again, where a determination is made that electrode 6 is dissolving or otherwise eroding at an unacceptably high rate, the focusing of the currents can be adjusted to the arrangement also seen in the middle or two another arrangement for that matter.

In an embodiment, there is a method of and a device for executing voltammetry and obtaining data based on that voltammetry within a human, such as within a cochlea of a human and evaluating the obtained data and implementing a treatment based on the evaluation. In an embodiment, the evaluation of the obtained data is the detection of a presence of and/or the determination of concentration of a substance, such as a therapeutic substance, such as a drug, in the human. In an embodiment, the evaluation of the obtained data can be the concentration of the substance in an electrolyte (e.g., cochlear fluid/perilymph). The substance could be blood, or any of the substances detailed herein. The substance could be biomarkers indicative of/byproducts of an infection for example. The substance could be dopamine or other neurotransmitters.

In an embodiment, these substances can be chemicals produced by an underlying ailment or an underlying scenario, which chemicals can be detected utilizing voltammetry. Because voltammetry can be utilized to identify specific chemicals, such as for example identifying local maxima and/or minima on a CV curve, the techniques herein can be utilized to identify specific chemical, and thus deduce an environment within the human. Based on this deduction, actions can be taken, such as, for example, the application of a therapeutic substance and/or the regulation of future application of a substance (e.g., if the concentrations of a given drug are deemed to be sufficiently high, less of that drug would be given, whereas in an alternative scenario, if the concentrations are deemed to be too low, or that drug would be given, with these concentrations can be determined utilizing voltammetry techniques detailed herein).

Accordingly, embodiments can utilize voltammetry to develop data upon which a treatment can be based, started, or modified, or halted, accordingly.

With respect to the aforementioned chemicals that can be the byproducts of infection for example, or chemicals that can represent a chemical's signature of blood for example, additional actions can be taken. For example, if the chemical signature obtained from the voltammetry indicates an infection, and anti-biotic or the like can be provided. If the chemical signature obtained from the voltammetry indicates blood in the cochlea for example, it can be determined that the implantation technique caused some form of trauma, such as, for example, scala puncture, and the electrode array could be repositioned in short order by way of example. With respect to the latter, in some embodiments, the voltammetry is executed during and/or shortly after insertion of the electrode array into the cochlea. As noted above, embodiments can include executing voltammetry utilizing the implant during a surgical technique. If for example the chemical signature indicates blood, remedial actions can be taken. While the just detailed remedial action given as an example in tail repositioning of the electrode array, in an alternate embodiment, an antibacterial agent could be preemptively provided into the cochlea, or a clotting agent could be provided, etc.

Further with respect to the coatings on the electrodes detailed above, in an exemplary embodiment, the coatings could aid in the detection of the given chemical signatures that are desired to be searched for utilizing voltammetry. A given coating could be provided for a certain drug, for example. A coating on one electrode could be for one drug, any coating on another electrode could be for another drug. There could be a coating on another electrode to detect the chemical signature for blood, and a coating on another electrode to detect for infection. One or more of these coatings could be bioresorbable, and the rate of bioresorption could be different. For example, the coating to detect the chemical signature of the byproducts of infection could be by a resort faster than the coating for a drug that will be provided for weeks or months. And again, note that in some embodiments, an entire channel or two or more could be sacrificed for the purpose of being able to detect a chemical signature of a certain compound, if such is deemed sufficiently utilitarian or otherwise sufficient to outweigh the elimination of that channel during the life or a large portion of the life of the implant.

Briefly, in an embodiment, the implemented treatment is the administration of a therapeutic substance to the human. In an embodiment, the action of implementing the treatment is an action that would not be executed if the evaluation did not indicate the increase. In an embodiment, the implemented treatment is the administration of a therapeutic substance to the human. In an embodiment, the implemented treatment is the execution of an optional cleaving of a prodrug previously provided to the cochlea. In an embodiment, the “treatment” is the continuing, halting and/or adjusting the implanted treatment based on the evaluation. In an embodiment, the implemented treatment includes prescribing one or more of systemic steroids, anticoagulants, clot busters, antifibrotics, antiproliferatives or NSAIDs. In an embodiment, instead of implementing the treatment, there is increasing an aggressiveness of a treatment or decreasing an aggressiveness of the treatment.

Thu, in an embodiment, there is a method that includes the method action of analyzing data based on the executed voltammetry and based on the analysis, prescribing a substance to be ingested by the human to address a phenomenon identified from the analysis and/or proscribing a substance to be injected by the human to address the phenomenon. This could be any of the therapeutic substances herein, including drugs, steroids, etc. In an embodiment, there is a method that includes the method action of analyzing data based on the executed voltammetry and based on the analysis, prescribing a substance to be ingested by the human to address a chemical phenomenon associated with body fluid of the human identified from the analysis and/or proscribing a substance to be injected by the human to address the chemical phenomenon. In an embodiment, there is a method that includes the method action of analyzing data based on the executed voltammetry and based on the analysis, providing a substance to the human via an implanted medical device implanted in the human to address a chemical phenomenon associated with body fluid of the human identified from the analysis. In this regard, in an exemplary embodiment, the implantable component can be “pre-charged” with a therapeutic substance. In this exemplary embodiment, the therapeutic substance can be delivered on an as-needed basis. If the voltammetry indicates that there is no “problem” the therapeutic substance would not be delivered, or otherwise can be delivered more slowly relative to that which would otherwise be the case (in some scenarios, it will be ultimately necessary to dispense with the therapeutic substance so as to avoid a scenario where there is a sudden release that is unintended, and thus tiny negligible amounts could be released over time, thus spreading out the delivery of the therapeutic substance and thus rendering any affected by that substance negligible). If there is a problem based on the voltammetry, the therapeutic substance will be delivered in a controlled manner.

It is also noted that while some embodiments herein are disclosed as being under the ultimate control of a human, such as where the human analyzes the data associated with the voltammetry, and then implements a course of action, in other embodiments, all of this could be executed automatically. In an exemplary embodiment, the medical device can be configured to execute voltammetry and then analyze the voltammetry and then take corrective actions based on the analysis.

It is briefly noted that while embodiments herein have been directed towards the utilization of devices that have a current source to implement the voltammetry techniques herein, in other embodiments, when not specifically noted, a voltage source can be utilized. In this regard, the utilization of a current source has utilitarian value in that existing cochlear implants include such and thus no new hardware is needed to implement voltammetry in at least some exemplary embodiments. However, other embodiments utilize a voltage source, such as, for example, the above-noted handheld tool could use such, although in some other embodiments, the handheld tool also utilizes a current source. In an exemplary embodiment, the above-noted handheld tool is implemented utilizing hardware of the cochlear implant, release the electronics package thereof, and simply incorporated into a handheld tool. Put another way, the medical devices that are not implants can be medical devices that are jumpstarted with respect to their design and manufacture by utilizing existing products, such as cochlear implant electronic devices.

A utilitarian result of the teachings detailed herein is that one or more stimulation channels of a cochlear implant, by way of example, can be continued to be utilized beyond that which would otherwise be the case if the deleterious wear event were to occur (the maintenance can be for any one or more of the temporal periods noted above with respect to the electrode). Further, the teachings detailed herein can be utilized to determine why certain channels will no longer provide utilitarian stimulation (and in some embodiments that the channels are no longer providing utilitarian stimulation), thus enabling workaround channels to be developed by adjusting the electrodes that are utilized to provide stimulation, where electrodes that still have structural effectivity for stimulation can be utilized to a degree greater than that which would otherwise be the case so as to compensate for the now defunct electrodes of the underlying initial channels.

In an exemplary embodiment, there is a medical device that includes electrodes, such as, for example, a cochlear implant, a sleep apnea device that utilizes implanted electrodes, a retinal prosthesis, a spine stimulator, a pacemaker, and epilepsy monitoring device, and epilepsy treatment device (that uses electrodes to provide stimulation to the brain with the nerves), a vagus nerve stimulator, an EKG monitor, an EEG monitor, a heart stimulator, etc. In this exemplary embodiment, the medical device includes an implantable portion of the medical device, the implantable portion including at least one electrode, including a plurality of electrodes, including any one or more of the numbers of electrodes detailed above. Further, the implantable portion is configured to, while implanted in a human, obtain data indicative of wear and/or passive dissolution and/or active dissolution of at least one of the electrodes of the plurality of electrodes.

At least some of the teachings detailed herein, as can be inferred, are applicable to active, as opposed to passive, implantable medical devices, such as by way of example only and not by way of limitation, pain stimulators, vestibular stimulators, deep brain stimulators, cardiac devices, etc. other embodiments are directed towards passive implantable medical devices.

In an exemplary embodiment, the implantable portion is the internal component(s) of FIG. 1E. The electrodes can be the electrodes 148 of FIG. 2A and/or FIG. 2B. In an exemplary embodiment, the implantable portion corresponds to a receiver-stimulator of the cochlear implant. In an exemplary embodiment, the receiver stimulator can have a logic circuit that can be configured to control the application of electrical signals to the various pertinent electrode(s) so as to provide voltage differentials in a controlled manner between electrodes. Alternatively, and/or in addition to this, the logic circuit can be in the external component of the cochlear implant, and this logic circuit can control the application of the pertinent electrical signals to the pertinent electrodes. Further, the implantable component can be configured to provide a telemetry signal to the external device indicative of voltage readings and/or current readings, etc., from the electrodes. In an exemplary embodiment, the telemetry signal can be the raw data, while in other embodiments, the telemetry signal can be a signal that is indicative of the results of an analysis that is executed by the implantable component.

In an exemplary embodiment, the cochlear implant can correspond to or otherwise be based on or otherwise be a modified version of the cochlear implant described in US patent application publication number 2012/0316454, published Dec. 13, 2012, entitled Electrode Impedance Spectroscopy, to the pioneer in impedance evaluation of cochlear implants, Paul Carter. In this regard, the device of the '454 publication can be utilized or otherwise modified so that it can be utilized to obtain data indicative of the passive dissolution and/or active dissolution of the at least one electrode. Other devices/methods that can be used or otherwise modified to obtain the data noted above are indicative of passive dissolution and/or motion or otherwise wear of the one or more electrodes are disclosed in the following patent application publications:
WO2018/173010, published Sep. 27, 2018, entitled Advanced Electrode Array Location Evaluation, to Inventor Nicholas Pawsey.
WO 2019/162837, entitled Advanced Electrode Data Analysis, published Aug. 29, 2019, to Inventor Paul Carter; and
WO 2019/175764, published September 2019, entitled Electrical Field Usage in Cochleas, to Inventor Ryan Melman.

The above-noted patent application publications disclose devices and methods that can be utilized or otherwise modified to obtain data relating to the various electrodes. Additional details of such will be described below, but it is noted that these various teachings utilize the existing electrodes of a cochlear implant electrode array, in combination with the electronics of the implant and/or the external component, to obtain electrical measurements relating to the electrodes. The signals applied to the electrodes can be modified to provide the stimulus that results in enablement of a phenomenon that can be read by read electrodes that corresponds to the obtained data. The point here is that the structure and the methods disclosed in those applications can be modified accordingly to implement the teachings detailed herein.

In an exemplary embodiment, consistent with the above-noted discussion regarding the telemetric features of at least some of the apparatuses disclose in the above noted publications, the implantable portion is configured to communicate the obtained data and/or data based on the obtained data transcutaneously to a device located outside the human. In an exemplary embodiment, this is achieved via the receiver stimulator, which includes an inductance coil, where the transceiver of the receiver stimulator is configured to provide a telemetric signal from the implant, through the skin of the human, to the external device (see FIG. 1A for example). This is the opposite of how the device normally works, where the external component captures sound, converts that sound into electrical signal that is applied to the inductance coil and the external component, which is transcutaneously communicated via an inductance link to the implant.

In some embodiments, the medical device is configured to analyze the obtained data and determine a wear status of the at least one electrode and communicate an indication of the wear status. In an exemplary embodiment, the wear status can be specifically a passive dissolution status and/or any active dissolution status of the electrode. In an exemplary embodiment, the analysis is executed by the external component, which can be in the form of a microprocessor or otherwise electronic circuitry with logic circuits configured to analyze the data and extract indicators (which will correspond to latent variables in at least some embodiments, again, more details of this below) that can be utilized to deduce the wear status of the electrode(s). The communication of the wear status or the other data detailed herein can be by way of a USB port on the external component or by way of a Bluetooth link with a remote device, or any other telemetric arrangement that can have utilitarian value with respect to communicating the wear status. The wear status can be stored in a memory, and this memory could be periodically accessed.

It is also noted that in some embodiments, it is the implantable portion that is configured to analyze the obtained data and determine a wear status of the at least one electrode and communicate an indication of the wear status. The pertinent electronics microprocessors can be located in the receiver-stimulator or another component of the medical device. (If in the implant, the data signal can be sent with the telemetry link noted above.)

And note that any one or more components can include a PID controller or can be configured to execute PID control to implement the voltammetry. A processor can be modified with firmware or software or the processor can have access to such, or another chip can have access to such.

In some exemplary embodiments, the medical device is configured to enable an adjustment of an operation of the cochlear implant to reduce a future passive dissolution and/or active dissolution rate of the at least one electrode and/or one or more other electrodes of the implantable component. Additional details of this will be described below, but by way of example only and not by way of limitation, in an exemplary embodiment, the medical device can be configured to enable the reduction of a pulse rate and/or a degree of focusing for some or all stimulation channels to reduce a stimulation amplitude associated with a given electrode relative to that which was otherwise the case before the adjustment.

It is also noted that in an exemplary embodiment, the medical device can be configured to analyze the obtained data and determine a wear status of the at least one electrode. That is, instead of simply being able to obtain the data indicative of wear of the at least one electrode, the medical device can be configured to actually use that obtain data for the utilitarian purpose just noted. This can be in combination with the device that is configured to enable the adjustment, or, in other embodiments, this can simply be a standalone feature that simply provides a warning or otherwise provides an indication that the electrode(s) (any reference to an electrode corresponds to a disclosure of an alternate embodiment of two or more electrodes or all of the electrodes of the medical device and was otherwise noted) is experiencing a passive dissolution and/or an active dissolution phenomenon that could be problematic in the short and/or long term. Indeed, in an exemplary embodiment, the wear status can simply be that there is a deleterious event that is occurring or otherwise will occur that will ultimately result in a potential problem. This as opposed to, for example, other embodiments, where the wear status is a percentage range of the electrode that remains for example (or a ballpark). Accordingly, such a specific wear status would be just that, a specific wear status. A wear status is a genus that encompasses the species of a specific wear status, which includes data that would enable one of ordinary skill in the art to reduce an approximate physical state of the electrode and/or deduce or otherwise estimate the remaining life of the electrode. Put another way, a wear status would be analogous to an indication that a tire pressure is low, and a specific wear status would be analogous to an indication that the tires are at between 65 and 75% of the pressure that they otherwise should be, etc.

All the above said, in an exemplary embodiment, the medical device is a relatively sophisticated device, which device is configured to analyze the obtained data and determine a specific wear status of the at least one electrode and automatically adjust an operation of the cochlear implant to reduce a future passive dissolution and/or active dissolution rate of the at least one electrode and/or one or more other electrodes of the implantable component. Again, as promised above, the adjustments that can be made will be detailed below. We further note that here, the device determines the specific wear status, as distinguished from the broader concept of the wear status. Also, we note that in this exemplary embodiment, the adjustment can not only extend the longevity or otherwise reduce the future passive dissolution and/or active dissolution rate of one electrode, such as the electrode from which the data is obtained, but can also extend the longevity or otherwise reduce the future passive dissolution and/or active dissolution rate of another electrode. In this regard, one or more of the electrodes can be utilized as test electrodes (they can also be fully functioning stimulating electrodes), which can be utilized as a proxy for the status of other electrodes. Further, in an exemplary embodiment, the device could make a determination that the electrode upon which the data is related is a “hopeless electrode,” and thus could make determinations to preserve the other electrodes. Thus, embodiments can include a medical device that is configured to analyze the obtained data and determine a wear status (which can include in some embodiments a specific wear status—wear status is a genus—a general wear status would exclude a specific sear status) of the at least one electrode and automatically adjust an operation of the cochlear implant to compensate for the wear of the at least one electrode. By way of example only and not by way of limitation, this could include shifting channels or implementing a constructive and/or destructive interference regime, such as that disclosed in US Patent Application publication 2010/0198301 to Zachary Smith, published Aug. 5, 2010, entitled Multi-electrode Channel Configurations, and/or US Patent Application publication No. 7,860,573 to Christopher van den Honert, published Dec. 28, 2010, entitled Focused Stimulation in a Medical Stimulation Device. Indeed, to be clear, in some embodiments, the unadjusted device operates according to one or more of the teachings of these two publications. It is that these publications enable the adjustments of the focusing of the stimulation in a manner that can account for the electrode that has suffered a deleterious event.

Of course, embodiments can be directed towards reducing the pertinent rates of both the electrode upon which the obtained data is based and one or more other electrodes.

In some embodiments, the medical device is configured to analyze the obtained data and determine a wear status of the at least one electrode and automatically recommend an action to reduce a future passive dissolution and/or active dissolution rate of the at least one electrode and/or one or more other electrodes of the implantable component and/or recommend an adjustment to an operation of the cochlear implant to compensate for the wear of the at least one electrode. With respect to the last feature, this was just described except with respect to the automatic adjustment. Here, the device simply recommends the adjustment. It is up to the user or a healthcare professional or a technician to implement the adjustment (which could be as simple as accepting the recommended adjustment). With respect to the former feature, this too has been discussed above, except with respect to automatic action to reduce the future passive dissolution and/or active dissolution rate. But to be clear, some embodiments can also utilize the determination and/or recommendation and/or analyses detailed herein to implement automatic action that alters stimulation parameters and/or the operation of the hearing prostheses, which automation can be executed by a software algorithm or any other computational regime that can enable such. This can be done automatically by the medical device and/or by a component in signal communication with the medical device, such as a handheld smart phone or the like, etc.

It is noted that while the above has been directed towards features of the medical device, it is noted that some of these features can exist alternatively and/or in addition to this in an external remote device that is remote from the medical device, such as by way of example only and not by way of limitation, a smart phone or smart device that is in signal communication with the medical device, such as by way of example only and not by way of limitation by a Bluetooth connection, or a computer such as a laptop or desktop computer that can be placed into signal communication with the medical device, and/or a device that is accessible via the Internet or the like which is located at or otherwise is embodied in a remote server that is tens of miles or more from the medical device. This is pertinent to the diagnostic and/or recommendation features detailed above. By way of example only and not by way of limitation, the actions of the analysis of the obtained data and the determination of the wear status and/or the recommended adjustment in operation, etc., can be executed by a remote computing device, such as a trained neural network by way of example, or any other computing device that can enable the teachings detailed herein. This data can be communicated to a healthcare professional for final approval, or can be communicated back to the user or the technician in control of the medical device. In an exemplary embodiment, as can be communicated directly to the medical device for appropriate implementation.

In at least some exemplary embodiments, the various measurements or otherwise data collection techniques detailed herein and/or variations thereof that have utilitarian value to implement the teachings detailed herein can be made on each electrode contact and/or a selection of contacts or otherwise a representative contact at regular intervals (daily, weekly, monthly, etc.) and/or irregular interviews and logged (e.g., by the implantable portion and/or the external portion of the prostheses and/or can be collected via a smartphone and/or a personal computer, etc.). This data can be provided to a data collection center or a data evaluation center in real time or at a later date. Changes in one of more of these measurements over time in a way that is known to be characteristic of active dissolution and/or passive dissolution of electrical contacts can be detected by a software algorithm running in the implantable portion and/or on the external portion of the prosthesis processor and/or in the aforementioned smart phone and/or PC, and/or at a remote data analysis center, which can be at a remote server as noted above. The algorithm may also consider data that is logged relating to the usage of the cochlear implant, such as, for example, the number of stimulation pulses and/or the current amplitude and/or the phase duration of pulses delivered over the life of the device through one or more or all contacts. Any operational parameter that can be logged that can be utilized to estimate or otherwise deduce the current wear state and/or a wear rate of an electrode can be utilized in at least some exemplary embodiments. The algorithm may also take into account the position of the contact on the array, the position of the array within the human, such as, for example, the position of a cochlear implant electrode array within a cochlear (which may be established though analysis of post-op x-ray and/or CAT scan), and/or the status of neighboring electrodes contacts. These spatially based features can impact the various measurements detailed herein. By taking into account the spatial variables, the accuracy of the data analysis can be further improved so as to further increase the accuracy of the analysis relating to the current wear rate and/or the current wear status of the electrode. The algorithm can then calculate an estimate of the wear status and/or wear rate of one or more or all of the electrode contacts on the array. This information can be delivered to a clinician who can decide whether changes to the prostheses, such as changes to stimulation parameters associated with a cochlear implant, are warranted in order to reduce the risk of premature wear out and/or otherwise to determine how to accommodate or otherwise address the fact that an electrode has effectively worn out. Alternatively, human evaluation can be utilized to evaluate the estimate of the wear status and/or wear rate of one or more or all of the electrode contacts of the array.

Any of the diagnostic and/or the determination and/or recommendation and/or analysis and/or adjustments detailed herein that are executed by the medical device can be executed by a laptop computer and/or desktop computer and/or a smart phone or smart device that is handheld and/or by server that is remote from the medical device accessible via the Internet was otherwise noted providing that the art enable such. It is also noted that in some embodiments, any of the diagnostic and/or determination and/or recommendation and/or analysis and/or adjustments detailed herein can be executed by a trained professional such as a healthcare professional or a technician unless otherwise noted providing that the art enable such.

Exemplary embodiments include management of life changes based on the voltammetry data. For example, in at least some exemplary embodiments, a stronger/higher magnitude current will be utilitarian as the recipient ages, where the increased current will result in a faster dissolution rate of the electrode. In an exemplary embodiment, an embodiment takes into account these changes, and proactively estimate the ultimate life expectancy of the electrode array. For example, all things being equal, statistical data based on the future hearing prostheses device needs of a recipient can be utilized to forecast future required for utilitarian settings of a given hearing prostheses. The teachings detailed herein can be utilized to provide an estimate of the remaining useful life of the electrode (taking into account, for example, a safety factor where an electrode must have at least 20 or 15% or so of its remaining mass or volume or thickness to be reliably useful). It could be that it is foreseen that the electrode will have to be explanted or otherwise changed out or otherwise will not last for its needed useful life. It could be that a remedial action now as opposed to in the future would be more utilitarian. By way of example, a surgery when a person is younger is typically “safer” than that which would be the case when the recipient gets older. In another exemplary embodiment, it could be that the electrode is “saved” 41 it is later needed. That is, for example, the performance of the implant could be reduced now so that higher performance could be utilized later, when it is needed. For example, the magnitude of current applied to a given electrode in the short run can be purposely reduced beyond that which would otherwise be the case and could provide a minor inconvenience, balance out by the fact that in later years, a higher current will be needed. The idea being that the electrodes are managed so as to achieve their desired otherwise needed life. Thus, embodiments disclosed herein include a management regime that can span years and/or decades, or any of the temporal periods detailed herein, where the actions taken are not only based on presents usages, but also can be based on future uses of the prostheses.

Some embodiments include testing or otherwise evaluating each of the electrodes of an implant. Conversely, some embodiments include testing only some of the electrodes out of the total number of electrodes. By way of example only and not by way of limitation, some embodiments will result in data being available that indicates which electrodes are utilized more than others in or otherwise which electrodes would statistically likely have a greater degree of wear relative to others. By way of example only and not by of limitation, the electrodes associated with speech frequencies would likely be experiencing greater wear than the electrodes for frequencies higher than the average speech frequencies. That said, in embodiments where, for example, the speech frequencies are spread out over a number of electrodes, and the higher frequencies are concentrated on a single electrode, the reverse can be the case. A noise or sound environment of the recipient can be evaluated. Data relating to the stimulation arrangements or otherwise the parameters of the implant can be evaluated. For example, if it is known that a given electrode experiences highly focused and/or high magnitude currents of short pulse durations, some embodiments include identifying that electrode as a leading candidate for early passive dissolution and/or active dissolution. Accordingly, at least some embodiments can include concentrating the evaluations on that electrode. Indeed, some embodiments can be such that it is not possible to evaluate or otherwise analyzed or otherwise monitor some electrodes. Still, in some embodiments, all of the electrodes can be monitored or otherwise analyzed.

At least some exemplary embodiments according to the teachings detailed herein utilize advanced techniques to analyze the data to forecast or otherwise ascertain a dissolution rate and/or a status of the solution, which are able to be trained or otherwise are trained to detect higher order, and/or non-linear statistical properties of the data inputted into the system, which data can correspond to any one or more of the examples detailed herein and/or any other that can have utilitarian value to ultimately forecast or otherwise ascertain or estimate a rate of passive dissolution/active dissolution and/or a status of the passive dissolution/active dissolution. An exemplary data processing technique is the so called deep neural network (DNN). At least some exemplary embodiments utilize a DNN (or any other advanced learning data processing technique) to process data, which processed data is utilized to evaluate the electrodes according to the teachings herein. At least some exemplary embodiments entail training data processing algorithms to process data to implement at least some of the exemplary methods herein. That is, some exemplary methods utilize learning algorithms or regimes or systems such as DNNs or any other system that can have utilitarian value where that would otherwise enable the teachings detailed herein to analyze the data relating to the electrodes.

Embodiments include utilizing a so-called “neural network” that can be a specific type of machine learning system. Any disclosure herein of the species “neural network” constitutes a disclosure of the genus of a “machine learning system.” While embodiments herein focus on the species of a neural network, it is noted that other embodiments can utilize other species of machine learning systems. Accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Embodiments include method actions associated with processes to train DNNs so as to enable those DNNs to be utilized to execute at least some of the method actions detailed herein.

It is noted that in at least some exemplary embodiments, the DNN or the product from machine learning, etc., is utilized to achieve a given ability to evaluate/process the data detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network.

All the above said, in some embodiments, standard processors that are programmed in the traditional manner/that are not machine learning based and/or chips that are formatted in a traditional manner and include logic circuitry that are configured to execute at least some of the exemplary method actions detailed herein are utilized. Computers that are programmed or otherwise configured to accept the data and/or retrieve the data and/or process the data or otherwise evaluate the data can be utilized to execute at least some of the method actions detailed herein. Note also that any reference to a method action herein that is implemented utilizing artificial intelligence and/or a neural network and/or machine learning corresponds to an alternate embodiment where the reference is to a functionality of a device. By way of example only and not by way of limitation, in an exemplary embodiment, if there is a disclosure herein of a medical device that is configured to evaluate the data relating to the electrodes and determine a wear rate and/or wear status of the electrodes, such corresponds to a disclosure of utilizing a product of machine learning to analyze that data, where the product of the machine learning can be a computer chip for example, which computer chip is part of the medical device.

It is noted that any method action disclosed herein corresponds to a disclosure of a non-transitory computer readable medium that has program there on a code for executing such method action providing that the art enables such. Still further, any method action disclosed herein where the art enables such corresponds to a disclosure of a code from a machine learning algorithm and/or a code of a machine learning algorithm for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A medical device, comprising:
an implantable portion of the medical device, the implantable portion including at least one electrode, wherein
the implantable portion is configured to, while implanted in a human, obtain data indicative of a state of a metal-electrolyte interface of the at least one electrode.

2. The medical device of claim 1, wherein:
the obtained data is based on a chemical reaction that takes place owing to energizement of the at least one electrode independent of voltage across the circuit that includes the electrode.

3. (canceled)

4. The medical device of claim 1, wherein:
the medical device is configured to obtain the data by generating a controlled current and measuring voltage.

5. The medical device of claim 1, wherein:
the medical device includes a control system that varies the current applied to the electrode to generate a desired voltage.

6-8. (canceled)

9. The medical device of claim 1, wherein:
the medical device includes a control system that varies the current applied to the electrode to generate an approximation to a constantly ramping voltage.

10. The medical device of claim 1, wherein:
the medical device is configured to vary a voltage source thereof to vary a voltage applied to the at least one electrode; and
the data is voltammetry data.

11. The medical device of claim 1, wherein:
the obtained data is based on a chemical reaction that takes place owing to energizement of the at least one electrode by maintaining a fixed voltage independent of a load resistance and/or an output current.

12. A medical device, comprising:
at least one electrode; and
electronics configured to controllably apply an electrical signal to the electrode, wherein the medical device is configured to enable in vivo obtention of data indicative of a chemical in an environment of the at least one electrode via voltammetry.

13. The medical device of claim 12, wherein:
the medical device includes a plurality of electrodes of which the at least one electrode is apart and a carrier carrying the plurality of electrodes; and
the electronics are configured to controllably apply respective electric signals to respective electrodes of the plurality of electrodes to stimulate tissue of a human.

14. The medical device of claim 11, wherein:
the medical device is a cochlear implant.

15. The medical device of claim 13, wherein:
the at least one electrode includes a coating that is bioresorbable, which coating influences the voltammetry in the presence of certain substances; and
the coating is bioresorbable and configured so that, upon bioresorption, the at least one electrode is usable to stimulate the tissue of the recipient.

16. The medical device of claim 13, wherein:
the at least one electrode includes a coating that is bioresorbable, which coating influences the voltammetry.

17-18. (canceled)

19. The medical device of claim 12, wherein the medical device is configured to vary non-zero current applied to respective electrodes by over 2 mA within 5 microseconds.

20. The medical device of claim 12, wherein the medical device is a non-implant medical device.

21-41. (canceled)

42. A medical device, comprising:
an implantable portion of the medical device, the implantable portion including at least one electrode, wherein
the implantable portion includes a current source, and has as its principle of operation controlling current from the current source to stimulate tissue of a human to evoke a reaction associated with the tissue, and
the medical device is configured to control current from the current source to establish a specific electrical phenomenon in reaction to the control of the current source and obtain data based on the electrical phenomenon, which electrical phenomenon and data is sufficient to execute voltammetry.

43. The medical device of claim 42, wherein:
the medical device includes a control system configured to approximate an ideal cyclic voltammetry system using hardware of a current source.

44. The medical device of claim 42, wherein:
the medical device is configured to make rapid measurements of voltage at the electrode or another electrode and, based on the measurements, vary current magnitude and/or temporal length of application of current to approximate an ideal cyclic voltammetry system.

45. The medical device of claim 42, wherein:
the medical device is of a design where a software change can completely eliminate the ability to establish the specific electrical phenomenon.

46. The medical device of claim 42, wherein the implantable portion is configured to output data based on data indicative of temporally correlated values of current supplied to the electrode from the current source and temporally correlated voltage readings of at least 100 values and at least 50 readings per second.

47. The medical device of claim 42, wherein the implantable portion includes an electrode that is separate from electrodes used by the device in normal stimulative operation, which electrode that is separate is a reference electrode, and wherein the separate electrode has a relatively stable rest potential, and wherein the separate electrode is coupled to a high input impedance measurement amplifier.

48-50. (canceled)
